(19) United States
(12) Patent Application Publication
(10) Pub. No.: US 2011/0189319 A1
(43) Pub. Date: Aug. 4, 2011

(54) Lifeforce liquid supplement

(76) Inventors: Lorn Leitman, Key Biscayne, FL (US); Gustavo Barni, Pompano Beach, FL (US)

(21) Appl. No.: 12/658,169

(22) Filed: Feb. 4, 2010

(51) Int. Cl. A61K 36/55; A61K 36/00; A61P 43/00; A23L 1/30

(52) U.S. Cl. 424/768; 424/725; 426/2

(57) ABSTRACT

Two very important factors in human life—which play vital roles in promoting well being—includes returning the body to homeostasis and giving boost to body called lifeforce to get overall health free from diseases, i.e., its optimal balanced functioning ability. When the body is at homeostasis, there is no place for disease. It is only when the body is out of balance (something is deficient, in excess or stagnating) that pain, illness or disease can occur. The application described is based on returning the body to homeostasis (removing the problem and balancing the body) by providing lifeforce and not on dealing with the symptom (e.g., pain, vitamin deficiency). Imbalance results in various diseases and adverse health conditions (starting from aging to diabetes and cancers). This wonderful composition will interact with the body in a way that allows it to boost lifeforce and reach homeostasis no matter which direction it was. In this combination, in the form of liquid, all the 14 ingredients (Lepidium meyenii (Maca), Croton planstigma (Dragon's blood) tree sap, Uncaria tomentosa (Cat's Claw), Morinda citrifolia (Noni fruit) 4:1 PE, Lutein, Lycopene 5%, Flaxseed Oil (Omega-3-Fatty Acids), Vinpocetine, Phosphatidyl Serine 50%, Korean Ginseng 80%, Bacopa monnieri (Bacopin), CDP Choline (Cognizing), Guaranine (Guarana Seed PE 12%), Yerba Mate Ext. 8%) work as mixture for returning the body to homeostasis and give it lifeforce.

### INTRODUCTION

### 1. Technical Field

The invention of this product is related to deliver energy to cellular system and balance to vital systems and organs. It supports the brain, skin, eyes, immune, circulatory, antioxidant and energy systems.

### BACKGROUND OF THE INVENTION

The present invention relates to the field of pharmaceutical formulation science as well as the field of therapeutic applications of homeostasis. In particular, the invention relates to the components of liquid which helps energizing the cells and makes a balance to important systems in the body for a prolonged duration.

### BRIEF SUMMARY OF THE INVENTION

The present invention relates to liquid supplement that contains fourteen components acting as lifeforce and balance enhancers. In humans, consumption of this liquid results in significant protection against general weakness and imbalance of vital organs. As this liquid contains different components when compared to other rejuvenating and balance combinations, the level of action produced by this is substantially effective, and intense.

All The fourteen ingredients—Lepidium meyenii (Maca), Croton planstigma (Dragon's blood) tree sap, Uncaria tomentosa (Cat's Claw), Morinda citrifolia (Noni fruit) 4:1 PE, Lutein, Lycopene 5%, Flaxseed Oil (Omega-3-Fatty Acids), Vinpocetine, Phosphatidyl Serine 50%, Korean Ginseng 80%, Bacopa monnieri (Bacopin), CDP Choline (Cognizing), Guaranine (Guarana Seed PE 12%), Yerba Mate Ext. 8%—have two common properties; these ingredients work as potent energy boosters and overall well-being of the body. Apart from that, each of the ingredients has some medicinal uses as well.

The present invention has been carried out in order to test the activity of homeostasis liquid on humans. Therefore, the first aspect of the present invention relates to a composition intended for liquid that works as a potent lifeforce and homeostasis enhancer.

Second aspect of the invention relates to a method of treating diseases and symptoms associated with general imbalance and exhaustion in men and women. For example, methods of treating aging, age-related disorders, cardiovascular ailments (such as coronary artery disease) attributed to oxidative damages, infections and malignancies attributed to poor immunity, and so on.

The main functions of each component are:

Lipidium Meyenii: Lipidium Meyenii (Maca) has been used for centuries to increase vitality, promote libido, fertility and sexual performance and stamina. It is also used to relieve the symptoms of menopause and premenstrual syndrome (PMS), as well as enhance energy and strength during athletic performance.

Lycopene: Lycopene is a very efficient antioxidant, which can neutralize oxygen derived free radicals. Lycopene is generally known for its protective action against prostate cancer.

Croton planstigma (Dragon's blood) tree sap: It is used for pain control, relief from skin irritations, wound healing, stomach and bowel ailments.

Uncaria tomentosa (Cat's Claw): Uncaria tomentosa is used to stimulate the immune system, reduce inflammation, inhibit tumor growth, act as an anti-viral, enhance DNA repair on a cellular level and even improves memory.

Lutein: Lutein belongs to category carotenoids which are powerful antioxidants linked to heart health, regulation of blood sugars (diabetes) and cancer prevention and reduction.

Noni: Noni acts as a potent immune-booster, antioxidant and anti-inflammatory agent. It also acts as an anti-tumor agent.

Flaxseed oil: Flaxseed oil is uded for prevention or control of dry eyes and to lower risk of heart disease and a reduction of chronic inflammation that can lead to a variety of serious diseases, including cancer and stroke.

Guaranine: Guaranine stimulates the central nervous system, increases metabolism, and has a mild diuretic effect.

Bacopa monnieri (Bacopin): Bacopa is a great neurotonic, immuno-modulator, tranquilizing, memory and learning enhancing, cerebral activator, anti-ulcer, antispasmodic, anti-asthmatic herb.

Vinpocetine: Vinpocetine is antioxidant and has cerebral blood-flow enhancing and neuroprotective effects, and is used as a drug in cerebrovascular disorders and age-related memory impairment.

Phosphatidyl Serine: Phosphatidyl Serine is a phospholipid which is vital to the structure and functioning of cells, and in humans it is vital to proper brain cell function and brain operation.

Korean Ginseng: Korean Ginseng stimulates the formation of blood vessel and improves blood circulation in the brains, thereby improving memory and cognitive abilities. It is also used for diabetes, migraine, infections, radiation and chemotherapy protection and to stimulate the appetite.

Yerba Mate Ext. 8%: Yerba Mate Ext. 8% is hypocholesteremic (cholesterol lowering), antioxidant, hepato protective (protecting the liver) properties. It has rejuvenating, nutritional, and energizing effects, particularly for mental and physical fatigue

CDP Choline (Cognizin®): Citicholine helps make phosphatidylcholine in human brain cell membranes in older individuals; may increase acetylcholine synthesis; improves mental performance in patients with Alzheimer's disease; and even improves memory in elderly patients with memory deficits.

### DESCRIPTION OF SPECIFIC EMBODIMENTS

The Maca (Lepidium meyenii walp), is an ancient potent reserving root considered a superfood due to its excellent and scientifically proven properties. Maca root is an aphrodisiac, balances hormones, fights impotence, stress, insomnia, osteoporosis and more. Maca root is considered by peruvians to enhances fertility, boosts energy and promotes correct hormonal balance, Maca root also is considered as a PMS treatment and that improves Prostate Health.

Lycopene is a very efficient antioxidant, which can neutralize oxygen derived free radicals. The oxidative damage caused by these free radicals has been linked to many degenerative diseases such as cardiovascular diseases, premature aging, cancer and cataracts. In many countries it is legally allowed to advertise foods containing tomato lycopene as “containing antioxidants for the maintenance and support of healthy cells”. Lycopene is generally known for its protective action against prostate cancer.

Externally Croton planstigma sap is employed as an antiseptic, as a wound-healing agent, and for skin disorders. Internally, it is used for hemorrhaging, mouth and throat ulcers/infections, and intestinal disorders.

Uncaria tomentosa (Cat's Claw) is reputed to be a remarkably powerful immune system booster and effective in treating a wide array of maladies including cancer, systemic candidiasis, genital herpes, and AIDS. It also has anti-tumor, anti-inflammatory, and anti-oxidant properties. It has proven useful in treating arthritis, bursitis, allergies and numerous bowel and intestinal disorders. Anecdotal evidence indicates effectiveness in relieving side effects of chemotherapy.

Lutein is a possible Anti-cancer agent and it helps to ward off the effects of bad cholesterol, whilst also keeping arteries clear. It also helps combat arthritis and osteoarthritis aches along with the health of eyes.

Noni, also known as Indian Mullberry (Morinda citrifolia), is a tropical plant. The leaves, bark, root and fruits of the plant are used as traditional remedy for many diseases. Noni acts as a potent immune-booster, antioxidant and anti-inflammatory agent. It also acts as an anti-tumor agent.

Flaxseed oil is useful for healing scars, abrasions and also has anti-inflammatory properties and so is useful for inflamed skin or skin disorders such as psoriasis and eczema. Flax seed oil is also useful for dry skin.

Guaranine stimulates the central nervous system, increases metabolism, and has a mild diuretic effect. It has been suggested that in combination with other herbs, guarana (guaranine) may be effective in helping to promote weight loss. This effect is probably due to an appetite suppressing mechanism, an increase in calorie burning, or both.

As a potent nerve tonic, Bacopa Monnieri is used to restore, energize, nourish and strengthen the central nervous system. It is used to calm the nerves and act as an herbal anti-depressant, alleviating nervous exhaustion, anxiety, temporary depression and headaches. It has also been used to treat nervous breakdown, attention deficit and nervous deficit, due to injury or stroke.

Vinpocetine is a semi-synthetic derivative of vincamine. Vincamine is an alkaloid derived from the plant Vinca minor L., a member of the periwinkle family. Vinpocetine, as well as vincamine, are used in Europe, Japan and Mexico as pharmaceutical agents for the treatment of cerebrovascular and cognitive disorders. It is sometimes called a nootropic, meaning cognition enhancer, from the Greek noos for mind.

While its mechanisms of action are unknown, Phosphatidyl Serine is thought to maintain nerve cell integrity, enhance neurotransmitter signal efficiency, enhance cognition, elevate mood, enhance memory, increase the brains absorption of nutrients, and improve nerve cell signal transmission. Phosphatidylserine is a naturally-occurring molecule important for brain function. Although the body can make its own phosphatidylserine, most of the nutrient is obtained through the diet. Phosphatidylserine is also available in supplement form. Phosphatidylserine supplements are sometimes claimed to be beneficial for the age-related cognitive decline (problems in mental functioning, such as memory loss), Alzheimer's disease or other forms of dementia, Attention deficit hyperactivity disorder (ADHD) and depression. Phosphatidylserine is also used for improving mental functioning in young people and improving athletic performance.

Studies have found that Korean ginseng improves performance and stamina, as well as reaction times in the elderly. It also is thought to have mild effects on the central nervous system.

Korean ginseng can have beneficial results on the mood and energy levels of anyone who takes it in the recommended amounts.

Yerba Mate Ext. 8% gives your brain a boost without making you nervous. It makes feel alert and sharp. Interestingly, it actually works as a tonic for the central nervous system, calming the body and the mind. It has also been shown to improve mood and concentration, reduce anxiety, and prevent mental fatigue.

Citicoline has beneficial physiological actions on cellular function that have been extensively studied and characterized by mechanism. Citicoline has been found to support membrane integrity, enhance acetylcholine formation, and to contribute to such critical metabolic functions as nucleic acids (e.g., RNA and DNA) and protein synthesis. In the brain, in addition to promoting phospholipid synthesis, citicoline also inhibits phospholipid degradation.

### BRIEF DESCRIPTION OF THE SEVERAL VIEWS OF THE DRAWINGS

Not Applicable

### DETAILED DESCRIPTION OF THE INVENTION

The detailed descriptions of 14 components are as follows:

### Description

Yerba mate Extract 8%

Yerba mate is a species of holly (family Aquifoliaceae) which is native to subtropical South America. Yerba mate plant is a shrub or small tree up to 15 meters tall. The leaves are evergreen, with a serrated margin. The flowers are four petal small and greenish-white.

Yerba mate is known to South Americans as the “Drink of the Gods” and has been consumed for centuries as a tonic and natural stimulant beverage. The infusion called mate is prepared by steeping dry leaves (and twigs) of yerba mate in hot water, rather than in boiling water like black tea. In Argentina, Uruguay, Paraguay and southern Chile drinking mate with friends from a shared hollow gourd with a metal straw is a common social practice.

Yerba Mate contains over 250 natural compounds, primarily alkaloids like caffeine, theophylline, and theobromine. These alkaloids act together and stimulate the nervous system and increase the metabolism. They also act as a diuretic, causing the body to shed water. Yerba Mate herb generally contains between one and two percent caffeine and around one half to one percent theobromine, the extract has been concentrated and standardized to 8% caffeine, with a significant increase in theobromine content as well. The use of this superb extract suppresses appetite, increases the burning of calories, increases urination, and is excellent for overall health.

### Actions and Pharmacology

### Actions:

Yerba mate extract can boost immunity, restore youthful hair color, retard aging, combat fatigue, control the appetite and eliminate insomnia”, and it has hypocholesteremic (cholesterol lowering), antioxidant, hepato protective (protecting the liver) properties. It has a bitter taste which is due to the phenolic constituents of the leaves. It has rejuvenating, nutritional, and energizing effects, particularly for mental and physical fatigue. It can be a effective weight-loss aid and scientific research shows mate to be a powerful antioxidant and that it can protect DNA from double-strand breaks. It also has the ability to inhibit LDL oxidation. Mate extract naturally contains a wide range of polyphenols, methylxanthines, and chlorogenic acid, which together are responsible for its many health benefits.

### Mechanism of Action:

Yerba mate has significant antioxidant activity. Yerba mate contains a higher content of flavonoids and caffeoyl derivatives than any other assayed species. It can protect DNA from double-strand breaks. It also has the ability to inhibit LDL oxidation. Yerba mate also provides sustainable energy due to its complex combination of xanthine alkaloids and caffeine. Although its caffeine content is comparable to coffee, the stimulation is balanced by yerba mate's nutritional content. The metabolic effects of mate appear to include the ability to maintain aerobic breakdown of carbohydrates during exercise for long periods of time. As a result, more calories are burned, thereby increasing cardiac efficiency and delaying the build-up of lactic acid. Additionally, mate's blend of xanthine alkaloids: caffeine, theophylline and theobromine, provide sustaining energy. Polyphenols found in yerba mate have been shown to prevent both the growth of bacteria responsible for bad breath and the bacteria's production of odorous compounds.

### Pharmokinetics

The details about the pharmacokinetics of Yerba mate are not available; however, it appears that it is administered orally and taken up for the metabolism from oral route.

### Indications and Use

Yerba Mate is recommended throughout South America for its rejuvenating, nutritional, and energizing effects, particularly for mental and physical fatigue.

### Research Summary

One study investigated the vasorelaxant properties of the aqueous and acid n-butanolic extractable fractions from yerba mate leaves. Perfusion pressure was evaluated using isolated and perfused mesenteric arterial beds (MABs) from rats fed hypercholesterolemic and standard diets. Extract-induced vasorelaxation in the presence and absence of various inhibitors was examined. These results suggest that yerba mate induces vasodilation in rats fed a standard diet in a dose-dependent manner and that the hypercholesterolemic diet substantially reduced the effect of yerba mate.

One study indicated that extracts of yerba mate tea (Ilex paraguayensis) inhibit the growth of the endothelial cells. The extract was fractionated and found to have novel cinnamate esters that inhibit proteasome activity. Based upon these findings, preclinical and clinical trials of topical cinnamate esters as proteasome inhibitors are warranted for psoriasis and other inflammatory disorders.

Researchers aimed to address the hypothesis that polyphenol-rich yerba mate extracts are capable of inhibiting advanced glycation end-products (AGEs) formation and to compare the potency of these extracts with green tea and with the standard antiglycation agent aminoguanidine. Glycation, the nonenzymatic adduct formation between sugar dicarbonyls and proteins, is one key molecular basis of diabetic complications due to hyperglycemia. They found that the results demonstrated a significant, dose-dependent effect of water extracts of yerba mate on AGE adducts formation on a protein model in vitro.

Researchers examined the effects of a yerba mate extract on post-ischemic alterations derived from 20 minutes of global ischemia and 30 minutes of reperfusion, isolated rat hearts were treated 10 minutes before ischemia and the first 10 minutes of reperfusion with yerba mate 30 microg/ml. In other hearts, chelerythrine (1 microM), a protein kinase C blocker, or 1(G)-nitro 1-arginine methyl ester (1-NAME), a nitric oxide synthase inhibitor, were administered prior to yerba mate infusion. The study concluded that these data are the first demonstration that yerba mate extract attenuates the myocardial dysfunction provoked by ischemia and reperfusion and that this cardioprotection involves a diminution of oxidative damage through a nitric oxide-dependent mechanism.

One study analyzed the antioxidant properties of yerba mate, by using two experimental models: the induction of DNA double-strand breaks (DSB) by hydrogen peroxide (H(2)O(2)) and lethality in Saccharomyces cerevisiae, as well as peroxide and lipoxygenase-induced human low-density lipoprotein (LDL) oxidation. They concluded that Yerba mate is a rich source of polyphenols and has antioxidant properties comparable to those of green tea which merit further in vivo intervention and cross-sectional studies.

In one study by researchers at the University of Illinois the in vivo and in vitro studies showed yerba mate to exhibit significant cancer-fighting activity. They found yerba mate to be “rich in phenolic constituents” and to “inhibit oral cancer cell proliferation” while it promoted proliferation of oral cancer cell lines at certain concentrations. This activity was due in part to inhibition of topoisomerase II activity in yeast.

Research also shows that yerba maté preparations can alter the concentration of members of the ecto-nucleoside triphosphate diphosphohydrolase (E-NTPDase) family, resulting in an elevated level of extracellular ATP, ADP, and AMP. This was found with chronic ingestion (15 days) of an aqueous yerba mate extract, and may lead to a novel mechanism for manipulation of vascular regenerative factors, i.e., treating heart disease.

The indigenous of South America traditionally use yerba mate to treat gastrointestinal disorders as eupeptic and choleretic agent. Research conducted by a team at Catedra de Farmacologia in Buenos Aires, Argentina found that yerba mate does in fact induce an increase in bile flow and enhance intestinal transit.

### Contradictions, Precautions, and Adverse Reactions

### Contradictions:

Yerba mate is contraindicated in patients with insomnia or problems sleeping, nervousness, stomach upset and irregular heartbeat. It inhibits the appetite as well.

### Precautions:

The body system can become mildly dependent on the caffeine in Yerba Mate. Withdrawal symptoms are mild and usually consist of headaches, tiredness and cravings.

### Adverse Reactions:

It can contribute to muscle tension when consumed in excess, especially for those who are prone to it. Also caffeine intoxication, the effects of which include rambling, confused thought and speech, insomnia, muscle twitching, and gastrointestinal issues can occur.

### Interactions:

It's not recommended to take Yerba Mate with other stimulant drugs or herbs such as ephedra or asthma medications, because adverse effects from those medications and supplements can be enhanced when combined with Yerba Mate.

Over-dosage: At present over dosage is not documented.

Dosage and administration: Sufficient literary evidence does not exist.

### CDP Choline (Cognizin®)

### Description

Citicoline (INN) is a psychostimulant and also known as cytidine diphosphate-choline (CDP-Choline) and cytidine 5′-diphosphocholine. Cognizin is the consumer brand for Citicoline produced by Kyowa Hakko Bio. Co., Ltd. and marketed as a dietary supplement and food ingredient by Kyowa Hakko U.S.A. It is an ingredient in some energy drinks such as “5-Hour Energy”. CDP-choline is approved in Europe and Japan for use in stroke, Parkinson's disease and other neurological disorders and is sold by the brand name Citicoline. CDP-choline can be taken as a more potent form of choline. Choline plays many roles in the body. Metabolically, it functions as a precursor for phosphatidylcholine biosynthesis, as a precursor for acetylcholine biosynthesis and as a methyl donor. It serves as a precursor for phosphatidylcholine and also of the phospholipid sphingomyelin. Phosphatidylcholine and sphingomyelin are structural components of biological membranes. These phospholipids also serve as precursors for the intracellular messengers ceramide and diacylglycerol. It is also the precursor of the signaling lipids, platelet-activating factor (PAF) and sphingosylphosphoryl-choline. It helps to improve focus and mental energy and may possibly be useful in the treatment of attention deficit disorder Choline, as well as other substances, such as methionine, folic acid and vitamin B₁₂, that prevent deposition of fat in the liver are known as lipotropes. The primary criterion used to estimate the Adequate Intake (AI) for choline is the prevention of liver damage as assessed by measuring serum levels of the liver enzyme alanine aminotransferase or ALT.

CDP-choline supplements increase dopamine receptor densities, and can ameliorate memory impairment caused by environmental conditions.

CDP-choline helps make phosphatidylcholine in human brain cell membranes in older individuals; may increase acetylcholine synthesis; improves mental performance in patients with Alzheimer's disease; and even improves memory in elderly patients with memory deficits.

Many studies have shown that citicoline prevents, reduces, or reverses the negative effects of a deficient blood supply in most human, animal and cellular models studied. Citicoline acts in head trauma models to decrease and limit nerve cell membrane damage, restore intracellular regulatory enzyme sensitivity and function, and limit edema. Considerable accumulated evidence supports the use of citicoline to enhance membrane maintenance, membrane repair, and neuronal function in traumatic conditions that injure the brain.

In rats with posttraumatic motor and spatial memory-performance deficits caused by traumatic brain injury, citicoline increased acetylcholine release in the dorsal hippocampus and neocortex. Eighteen days of citicoline administration resulted in significantly less cognitive deficits than injured saline-treated rats. Citicoline also lessened the memory-disrupting effects of scopolamine. Amazingly, a single-injected administration of citicoline increased extracellular levels of acetylcholine in dorsal hippocampus and neocortex in normal, awake, freely moving rats. It was concluded that spatial memory performance deficits are, at least partially, associated with deficits in central cholinergic neurotransmission and that treatments which enhance acetylcholine release (following traumatic brain injury) may lessen cholinergic-dependent neurobehavioral deficits.

Beneficial effects of exogenous citicoline also have been postulated and/or reported in experimental models for dyskinesia, Parkinson's disease, cardiovascular disease, aging, Alzheimer's disease, learning and memory, and cholinergic stimulation.

By activating the synthesis of critical components in cell membranes, citicoline boosts levels of neurotransmitters such as acetylcholine, and enhances cerebral energy metabolism. Citicoline can help preserve and protect proper memory structure and function. Thus, citicoline can be of significant value in helping to prevent age-associated cognitive impairment. It even boosts mitochondrial energy production, causing the re-absorption of cerebral edema, which can be caused by trauma or even stroke.

### Actions and Pharmacology

Actions: Stabilized CDP-Choline (cytidine 5′ diphosphocholine) is a naturally occurring, water soluble biological compound that is an essential intermediate for the synthesis of phosphatidylcholine, a major constituent of the grey matter of brain tissue (30%). CDP choline is metabolized to yield the free nucleotide cytidine and choline. Scientific research demonstrates that CDP Choline consumption promotes brain metabolism by enhancing the synthesis of acetylcholine, restoring phospholipid content in the brain and regulation of neuronal membrane excitability and osmolarity (by its effect on the ATP-dependent sodium and potassium pumps).

Mechanism of Action: This unique form of choline readily passes through the blood-brain barrier directly into the central nervous system. Once past the blood-brain barrier, CDP-choline activates the synthesis of critical components in cell membranes, boosts levels of neurotransmitters such as acetylcholine, and enhances cerebral energy metabolism.

By boosting mitochondrial energy production, CDP-choline causes the re-absorption of cerebral edema caused by trauma or stroke.

In studies carried out in patients with head trauma, CDP-choline accelerated recovery from post-traumatic coma and restoration of walking ability. The CDP-treated group demonstrated better functional results and reduced hospital stays. In patients with less-severe head trauma, CDP-choline improved cognitive and memory deficits.

Phosphatidylcholine is the major structural and functional component of brain cell membranes. Without phosphatidylcholine, brain cells lose their youthful function and suffer the degenerative structural changes associated with aging. CDP-choline is an essential intermediate in the conversion of choline to phosphatidylcholine and may be a key missing link in the quest to protect brain cells against age-related pathological degeneration.

### Pharmacokinetics

Citicoline is a water-soluble compound with greater than 90-percent bioavailability. Pharmacokinetic studies on healthy adults show oral doses of citicoline are rapidly absorbed, with less than one percent excreted in feces. Plasma levels peak in a biphasic manner, at one hour after ingestion followed by a second larger peak at 24 hours post-dosing. Citicoline is metabolized in the gut wall and liver. When taken orally, citicoline is metabolized in the gut to cytidine and choline. Both compounds are absorbed independently and taken up by brain cells. Within the brain cell cytidine is converted to cytidine monophosphate, cytidine diphosphate, and cytidine triphosphate. Choline is phosphorylated to form phosphocholine. Phosphocholine combines with cytidine triphosphate to reform citicoline. Citicoline then rapidly combines with diacylglycerol to from phosphatidylcholine. The byproducts of exogenous citicoline formed by hydrolysis in the intestinal wall are choline and cytidine. Following absorption, choline and cytidine are dispersed throughout the body, enter systemic circulation for utilization in various biosynthetic pathways, and cross the blood-brain barrier for resynthesis into citicoline in the brain.

Indications and use: May be taken with or without food.

### Research Summary

CDP-Choline is known to improve learning ability, retards the further progression of Alzheimer's Disease, increases blood Circulation and oxygen utilization within the Brain. It is used as a brain circulation stimulator to treat disturbances of consciousness that may follow brain injury (e.g. concussion) or brain surgery. CDP-Choline is a valuable co-therapy for Parkinson's Disease.

Ninety-two patients affected by chronic cerebrovasculopathy were treated with cytidine diphosphate choline (CDP-choline) 1000 mg/day i.m. or with placebo, in a double-blind study. Two cycles of therapy of 4 weeks each were performed, with an interval of 1 week. There were 46 patients in each group with chronic cerebrovascular diseases, and the two groups were comparable as far as mental deterioration was concerned. The comparison between the two groups revealed significant improvements in the CDP-choline group compared with the placebo group in some of the attention capabilities. No side-effects were detected in the CDP-choline group.

In one study the effect of intracerebroventricular (i.c.v.) administration of cytidine-5′-diphosphate (CDP) choline on plasma adrenocorticotropin (ACTH), serum growth hormone (GH), thyroid stimulating hormone (TSH), follicle stimulating hormone (FSH) and luteinizing hormone (LH) levels in conscious rats was investigated. The involvement of cholinergic mechanisms in these effects was also determined. In basal conditions, CDP-choline increased plasma ACTH levels dose- and time-dependently, but it did not affect the TSH, GH, FSH and LH levels. In stimulated conditions, i.c.v. administration of CDP-choline produced an increase in clonidine-stimulated GH, thyrotyropin-releasing hormone (TRH)-stimulated TSH, LH-releasing hormone (LHRH)-stimulated LH, but not FSH levels.

Researchers tested if CDP-choline is an intermediate in the biosynthesis of phosphatidylcholine, a phospholipid essential for neuronal membrane preservation and function and this would attenuate the process of neuronal aging. Three groups of male mice were used in this study. An adult 12-month-old group, a 24-month-old, and an old experimental group (OEG) were administered orally a solution of CDP-choline (150 mg/kg per day) from 12 up to 24 months. Experimental observations suggest that CDP-choline has a positive effect on memory (reference errors were attenuated), and hippocampal morphology resembled that of younger animals.

Citicoline (CDP-choline; cytidine 5′-diphosphocholine), a form of the essential nutrient choline, shows promise of clinical efficacy in elderly patients with cognitive deficits, inefficient memory, and early-stage Alzheimer's disease. CDP-choline has also been investigated as a therapy in stroke patients, although the results of trials to date are inconclusive. Produced endogenously, CDP-choline serves as a choline donor in the metabolic pathways for biosynthesis of acetylcholine and neuronal membrane phospholipids, chiefly phosphatidylcholine. The principal components of CDP-choline, choline and cytidine, are readily absorbed in the GI tract and easily cross the blood-brain barrier. Exogenous CDP-choline, as the sodium salt, has been researched in animal experiments and human clinical trials that provide evidence of its cholinergic and neuroprotective actions.

Findings presented at the American Stroke Association's 27th International Stroke Conference indicate that CDP-choline may be helpful in reducing damage after a stroke. The most common cause of a stroke is blockage of a blood vessel within the brain, which leaves a portion of the organ starved for blood and oxygen. Bleeding within the brain can also lead to stroke. Much of the brain damage is caused by a cascade of harmful chemicals released by dying cells.

Researchers at the National Institute of Neurological Disorders in Bethesda, Md., reported data on two trials of the supplement citicoline, sold in some health food stores or on the internet as CDP-choline. This nutrient is related to choline. One of the studies involved 41 patients who received 500 milligrams of cdp-choline daily for 6 weeks after having a stroke. In the other, 62 stroke patients took 2,000 mg of the drug every day for 6 weeks. The trials also included 111 patients who received an inactive placebo. The researchers gauged the CDP-choline's effectiveness by measuring the size of the infarct, or area of dead tissue. They scanned patients' brains within 24 hours of the stroke and again 12 weeks later. At the end of the study period, the investigators found that infarct size measured by MRI scans had increased by 85% in patients on placebo, by 34% in patients receiving the 500 mg dose of cdp-choline and by only 2% in patients receiving 2,000 mg cdp-choline.

In one study randomized, double-blind clinical trial was conducted to determine whether daily citicoline treatment improves neurocognitive and neuroimaging outcome over 12 months among patients diagnosed with vascular dementia (VaD). METHODS: 30 patients diagnosed with vascular dementia, were randomized and treated with either 500 mg of citicoline or placebo twice per day. RESULTS: The cdp-choline and placebo treatment groups did not differ in their neuropsychological performance at baseline and the 12-month follow-up. Significant declines in neuropsychological performance were noted, as well as significantly increased SH and reduced total brain volumes on MRI for both groups at the 12-month follow-up. It was concluded that the efficacy of long-term citicoline treatment for cognitive impairment and neuropathological decline in those patients already meeting criteria for VaD does not appear to be substantiated by the current study.

In one other study researchers investigated the efficacy and safety of the treatment with cdp-choline versus placebo in patients with Alzheimer disease. Thirty patients with mild to moderate senile dementia of the Alzheimer type were included in a double-blind, randomized and placebo-controlled clinical trial. After a 2-week period of drug washout, patients were treated with i) placebo or ii) 1,000 mg/day of CDP-choline for 12 weeks (84 days). Examinations were done at baseline (T0) and after the 12 weeks of treatment (T12). As compared to placebo, cdp-choline improved cognitive performance in Alzheimer's disease patients with APOE E4. CDP-choline also increased cerebral blood flow velocities in comparison with placebo when transcranial Doppler recordings from both hemispheres were considered together, as well as diastolic velocity in the left middle cerebral artery. Patients treated with cdp-choline showed an increase in the percentage of brain bioelectrical activity of alpha (occipital electrodes) and theta type (left side electrodes), accompanied by a decrease in relative delta activity particularly marked in the left temporal lobe. Treatment with cdp-choline tended to reduce serum IL-1 beta levels, mainly after 4 weeks of administration, with no modified blood histamine content. In addition, neither adverse side effects nor alterations in biological and hematological parameters were induced by cdp-choline. The present data indicate that cdp-choline (1,000 mg/day) is well tolerated and improves cognitive performance, cerebral blood perfusion and the brain bioelectrical activity pattern in AD patients. According to our results, it seems that cdp-choline might be a useful treatment in Alzheimer's disease, and that the efficacy of this compound is greater in patients with mild mental deterioration and/or bearing the epsilon 4 allele of the APOE.

### Contradictions, Precautions, and Adverse Reactions

Contradictions: Sometimes CDP-Choline can make can increase blood pressure in normal and hypotensive conditions.

Precautions: Insufficient reliable information available. Its use should be avoided during pregnancy and lactation.

Adverse reactions: The adverse reactions can be nausea and diarrhea. Some people who take citicoline also experience insomnia, palpitations, tachycardia, chest pain, hypotension, bradycardia.

Interactions: Methotrexate may reduce the pools of all choline metabolites. Choline, via its metabolism to betaine, works along with vitamins B₆, B₁₂ and folic acid in the metabolism of the homocysteine which is potentially atherogenic substance.

Over-dosage: Not Documented.

Dosage and administration: It can be administered orally and intravenously. Generally oral 1000-2000 mg/day administration is safe without any complications.

### Flaxseed Oil

### Description

Flax seed oil is a clear to yellowish oil obtained from the dried ripe seeds of the flax plant (Linum usitatissimum, Linaceae). It can polymerize and the reaction is exothermic, and rags soaked in it can ignite spontaneously. It is also called drying oil, which means that it hardens upon exposure to air. Flaxseed oil is a mixture of various triglycerides that differ in terms of their fatty acid constituents. These triglycerides are primarily derived from the following fatty acids: the saturated acids palmitic acid (about 7%) and stearic acid (3.4-4.6%), monounsaturated oleic acid (18.5-22.6%), doubly unsaturated linoleic acid (14.2-17%), triply unsaturated omega-3 fatty acid α-linolenic acid (51.9-55.2%). Food-grade flaxseed oil is cold-pressed, obtained without solvent extraction, and marketed as edible flaxseed oil. It contains high levels of omega-3 fatty acids, especially alpha-linolenic acid, which may be beneficial for reducing inflammation leading to atherosclerosis, preventing heart disease and arrhythmia, and is required for normal infant development. Plant breeders have developed flaxseed with higher alpha linoleic acid content (70%) and very low alpha linolenic acid content (<3%).

Flax seeds themselves contain lignans, a class of phytoestrogens considered to have antioxidant and cancer-preventing properties. In fact, flax seed oil is easily oxidized, and rapidly becomes rancid with an unpleasant odor unless refrigerated. Even when kept under cool conditions it has a shelf life of only a few weeks. Oil with an unpleasant or rancid odour should be discarded. Rancid oils contribute to the formation of free radicals and may be carcinogenic. Oxidation of flax seed oil is major commercial concern, and antioxidants may be added to prevent rancidification.

### Actions and Pharmacology

### Actions:

Omega-3 fats are used by the body to produce Series 1 and 3 prostaglandins, which are anti-inflammatory hormone-like molecules, in contrast to the Series 2 prostaglandins, which are pro-inflammatory molecules produced from other fats, notably the omega-6 fats, which are found in high amounts in animal fats, margarine, and many vegetable oils including corn, safflower, sunflower, palm, and peanut oils. Omega-3 fats can help reduce the inflammation that is a significant factor in conditions such as asthma, osteoarthritis, rheumatoid arthritis, migraine headaches, and osteoporosis. Flaxseed and flaxseed oil have been reported to possess cholesterol-lowering properties in laboratory studies. Human studies have used flaxseed products and measured effects on cholesterol, with mixed results. A recent human study found that dietary flaxseed significantly improved lipid (cholesterol) profile in patients with high cholesterol, and may favorably modify cardiovascular risk factors. Evidence suggests that people who eat an ALA-rich diet are less likely to suffer a fatal heart attack. ALA may reduce heart disease risks through a variety of biologic mechanisms, including platelet function (making them less “sticky”), inflammation, blood vessel health, and arrhythmia (irregular heart beat). Several human studies also suggest that diets rich in omega-3 fatty acids (including ALA) may lower blood pressure.

### Mechanism of Action:

Flaxseed is the most concentrated food source of the plant lignan, secoisolariciresinol, a precursor for enterolactone. It is thought that the abundance of these phytoestrogenic lignans contributes to its hormonal effect. Flaxseed has been shown to affect the length of the menstrual cycle in premenopausal women. In addition, flaxseed's inhibition of human breast cancer growth and metastasis in mice is due in part to its down-regulation of insulin-like growth factor I and epidermal growth factor receptor expression while its inhibition of prostate cancer in mice is attributed to its inhibition of cellular proliferation. Flaxseed may also alter estrogen metabolism, increasing the ratio of 2-hydroxyestrogen to 16 alpha-hydroxyestrone in a dose dependent fashion. The hormonal effects of flaxseed may also play a role in its ability to modulate prostate cancer biology and associated biomarkers and lower serum lipid levels. Flaxseed's renoprotective effects are thought to come from high concentration of alpha-linolenic acid, an omega-3 fatty acid precursor or through inhibition of angiogenesis, tyrosine protein kinases and cytokine-induced activation of transcription factors. Flaxseed may have laxative effect due to its fiber content.

### Pharmacokinetics

Dose-dependant urinary lignan response to ingested flaxseed has been observed. Processing flaxseed does not affect lignan absorption. Plasma lignan concentration was greater than baseline, nine hours after flaxseed ingestion. No plateau in serum lignan concentration was observable in dosages up to 25 grams.

### Indications and Use

It can cause increased bowel movements, constipation and flatulence.

### Research Summary

Alpha linolenic acid, the omega-3 fat found in flaxseed and walnuts, promotes bone health by helping to prevent excessive bone turnover-when consumption of foods rich in this omega-3 fat results in a lower ratio of omega-6 to omega-3 fats in the diet.

Other studies have shown that diets rich in the omega-3s from fish (DHA and EPA), which also naturally result in a lowered ratio of omega-6 to omega-3 fats, reduce bone loss. Researchers think this is most likely because omega-6 fats are converted into pro-inflammatory prostaglandins, while omega-3 fats are metabolized into anti-inflammatory prostaglandins. (Prostaglandins are hormone-like substances made in our bodies from fatty acids.)

Omega-3 fats are used to produce substances that reduce the formation of blood clots, which can reduce the risk of heart attack and stroke in patients with atherosclerosis or diabetic heart disease.

Omega-3 fats are also needed to produce flexible cell membranes. Cell membranes are the cell's gatekeepers, allowing in needed nutrients while promoting the elimination of wastes. While important for everyone, flexible cell membranes are critical for persons with diabetes since flexible cell membranes are much better able to respond to insulin and to absorb glucose than the stiff membranes that result when the diet is high in saturated and/or hydrogenated (trans-) fats. In the colon, omega-3 fats help protect colon cells from cancer-causing toxins and free radicals, leading to a reduced risk for colon cancer.

Individuals whose diets provide greater amounts of omega-3 fatty polyunsaturated fatty acids- and flaxseed is an excellent source of these essential fats-have lower blood pressure than those who consume less, shows data gathered in the International Study of Macro- and Micro-nutrients and Blood Pressure (INTERMAP) study. Average daily intake of omega-3 fatty acids was 2 grams. Participants with a high (0.67% kcal) omega-3 fatty acid percentage of their daily calorie intake had an average systolic and diastolic blood pressure reading that was 0.55/0.57 mm Hg less, respectively, than participants with lower intake. Previous research has found that a decrease of 2 mm Hg reduces the population-wide average stroke mortality rate by 6 percent and that of coronary heart disease by 4%.

Higher omega-3 fatty acid intake among the 2,238 subjects who were not using drugs, supplements, or a special diet for hypertension, heart disease, or diabetes was associated with a 1.01/0.98 mm Hg reduction in systolic and diastolic blood pressure, respectively.

For the 2,038 subjects in this group who did not have hypertension, greater intake was associated with a 0.91/0.92 mm Hg average systolic and diastolic reduction.

The researchers also found that omega-3s from nuts, seeds, and vegetable oils—such as walnuts and flaxseed—had just as much impact on blood pressure as omega-3s from fish.

In a study involving 40 patients with high cholesterol (greater than 240 mg/dL), daily consumption of 20 grams of ground flaxseed was compared to taking a statin drug. After 60 days, significant reductions were seen in total cholesterol, LDL cholesterol, triglycerides and the ratio of total to HDL cholesterol-in both groups.

Body mass index, total cholesterol, HDL-cholesterol, LDL-cholesterol, triglycerides, and the ratio of total cholesterol/HDL-cholesterol were measured at the beginning of the study and after 60 days.

In those eating flaxseed, significant reductions were seen in total cholesterol (−17.2%), LDL-cholesterol (−3.9%), triglycerides (−36.3%) and the ratio of total cholesterol/HDL-cholesterol (−33.5%) were observed in the diet+flax group, compared to baseline. Similar reductions were seen in those taking the statin. Benefits did not significantly differ between the two groups.

Greek researchers looked at the effect on systolic and diastolic blood pressure of a three-month trial during which 59 middle-aged men used either flaxseed or safflower oil in their daily diet.

Flaxseed oil is rich in the omega-3 fat, alpha-linolenic acid (ALA), which the body can metabolize into the cardioprotective long-chain omega-3 fatty acids, DHA and EPA, while safflower oil is a concentrated source of the omega-6 fat, linoleic acid (LA). The men received flaxseed oil supplying 8 grams of ALA daily or safflower oil providing 11 grams of LA per day.

At the conclusion of the 12-week study, both systolic and diastolic blood pressure was significantly lower in the men using the omega-3-rich flaxseed oil.

One possible explanation for this result is the anti-inflammatory effects of omega-3 fats. Both omega-6 and omega-3 fats are essential fatty acids: we need both types of fats to be healthy and must derive them from our food. Omega-6 fats, however, tend to promote excessive inflammation when not balanced by sufficient amounts of omega-3 fats in the diet.

A study published in the Archives of Internal Medicine confirms that eating high fiber foods, such as flaxseed, helps prevent heart disease. Almost 10,000 American adults participated in this study and were followed for 19 years. People eating the most fiber, 21 grams per day, had 12% less CHD and 11% less CVD compared to those eating the least, 5 grams daily. Those eating the most water-soluble dietary fiber fared even better with a 15% reduction in risk of CHD and a 10% risk reduction in CVD.

Flaxseed, a rich source of omega-3 fatty acids and lignans, put the brakes on prostate tumor growth in men who were given 30 grams of flaxseed daily for a month before surgery to treat their prostate cancer. The 40 men taking flaxseed, either alone or along with a low-fat diet, were compared to 40 men only following a low-fat diet, and 40 men in a control group who did not alter or supplement their usual diet. Men who took flaxseed, as well as those who took flaxseed combined with a low-fat diet did the best.

Eating about an ounce of ground flaxseed each day affects the way estrogen is handled in postmenopausal women in such a way that offers protection against breast cancer but will not interfere with estrogen's role in normal bone maintenance.

They do. Women whose diets provided the highest amounts of omega-3 fatty acids had a 17% lower risk of dry eye syndrome compared with those consuming the least of these beneficial fats.

In contrast, a diet high in omega-6 fats, but low in omega-3s, significantly increased DES risk. Women whose diets supplied a high ratio of omega-6 to omega-3 fatty acids had a 2.5-fold higher risk of DES syndrome compared to those with a more balanced intake of fatty acids.

### Contradictions, Precautions, and Adverse Reactions

### Contradictions:

Flaxseed may slow down the absorption of oral medications or other nutrients if taken at the same time. People with either diabetes or schizophrenia may lack the ability to convert ALA to EPA and DHA, the forms more readily used in the body. Therefore, those with either condition should obtain their omega-3 fatty acids from dietary sources rich in EPA and DHA.

Precautions: People with bleeding disorders shouldn't take it.

Adverse reactions: Flaxseed oil may cause breathing problems or tightness in your throat or chest and can cause chest pain. Also it can cause skin hives, rash, or itchy or swollen skin.

Interactions: Omega-3 fatty acids may increase the effects of blood thinning medications. Taking omega-3 fatty acid supplements may increase fasting blood sugar levels. Also taking omega-3 fatty acids during cyclosporine (Sandimmune) therapy may reduce toxic side effects, such as high blood pressure and kidney damage, associated with this medication in transplant patients.

Over-dosage: Doses of flaxseed oil of 30 grams per day and higher have been associated with loose stools and diarrhea.

### Dosage and Administration:

Lepidium meyenii (Maca)

### Description

Lepidium meyenii or maca is an herbaceous biennial plant or annual plant (some sources say a perennial plant) native to the high Andes of Bolivia and Peru. It is grown for its fleshy hypocotyl

(actually a fused hypocotyl and taproot), which is used as a root vegetable and a medicinal herb. Its Spanish and Quechua names include maca-maca, maino, ayak chichira, and ayak willku. The plant is considered a member of the species Lepidium meyenii, first observed and designated by Gerhard Walpers in 1843. In studying different specimens since the late 1960s, most botanists now consider the widely cultivated maca of today to be a newer domesticated species, L. peruvianum. This more recent designation was made by Dr. Gloria Chacon. The Latin name recognized by the USDA continues to be Lepidium meyenii, however most contemporary botanists employ the name “peruvianum” and consider it most accurate to describe the species”. The growth habit, size, and proportions of maca are roughly similar to those of the radish and the turnip, to which it is related. The green, fragrant tops are short and lie along the ground. Maca hypocotyls can be gold/cream, red, purple, black and green. Each is considered a genetically unique variety, as seeds of the parent plants grow to have roots of the same color. Recently, specific color strains have been exclusively propagated to ascertain their different nutritional and therapeutic properties. Cream colored roots are the most widely grown and are favored in Peru for their enhanced sweetness and size. Black maca is considered the strongest in energy-promoting properties, being both sweet and slightly bitter in taste. Red maca is also becoming popular with many people, and has been clinically shown to reduce prostate size in rats. These three ecotypes are the most commonly grown and exported. Hypocotyls grown from Peruvian seeds form with difficulty at low elevations, in greenhouses or in warm climates. Seeds obtained from Bolivian maca, which is native to lower altitudes, are more easily grown under such conditions. It is regarded as a highly nutritious, energy-imbuing food, and as a medicine that enhances strength, endurance and also acts as an aphrodisiac. During Spanish colonization maca was used as currency. In addition to sugars and proteins, maca contains uridine, malic acid and its benzoyl derivative, and the glucosinolates, glucotropaeolin and m-methoxyglucotropaeolin. The methanol extract of maca tuber also contained (1R,3S)-1-methyltetrahydro-carboline-3-carboxylic acid, a molecule which is reported to exert many activities on the central nervous system. The nutritional value of dried maca root is high, similar to cereal grains such as rice and wheat. It contains 60% carbohydrates, 10% protein, 8.5% dietary fiber, and 2.2% fats. Maca is rich in essential minerals, especially selenium, calcium, magnesium, and iron, and includes fatty acids including linolenic acid, palmitic acid, and oleic acids, and 19 amino acids, as well as polysaccharides. Maca's reported beneficial effects for sexual function could be due to its high concentration of proteins and vital nutrients, though maca contains a chemical called p-methoxybenzyl isothiocyanate, which reputedly has aphrodisiac properties.

### Actions and Pharmacology

### Actions:

Maca consists of 59% carbohydrate, 10% protein, 2.2% lipids and 8.5% fiber (dry weight). Maca contains 150 mg of calcium and 16.6 mg of iron per 100 g of dry matter. Although the following functions have not been proved in human studies, maca may: —enhance stamina-improve sexual function—regulate hormones. Because of its high fiber content, maca may also have a laxative effect.

### Mechanism of Action:

Maca is consumed as food for humans and livestock, suggesting any risk from consumption is rather minimal. It is considered safe to eat as any other vegetable food. However, maca does contain glucosinolates, which can cause goitres when high consumption is combined with a diet low in iodine. Darker colored maca roots (red, purple, black) contain significant amounts of natural iodine, a 10-gram serving of dried maca generally containing 52 μg of iodine. Though this is common in other foods with high levels of glucosinolate, it is uncertain if maca consumption can cause or worsen a goiter. Maca has been shown to reduce enlarged prostate glands in rats though its effects on humans are unknown.

### Pharmacokinetics

From animal studies and from limited human studies, it appears that Maca is absorbed from the gastrointestinal tract following its ingestion. However, the efficiency of its absorption, as well as its distribution, metabolism and excretion, are not well understood. Much research needs to be done in order to elucidate the pharmacokinetics of resveratrol in its various forms.

### Indications and Use

Maca has been used for male impotence, erectile dysfunction, menopausal symptoms and general fatigue. Maca can be used to increase libido and improve semen quality. It has been presumed that maca's hormone-normalizing effects may be due to the root's unique nutritional profile, which provides optimum levels of nutrients utilized by the body's endocrine system. In addition, maca may also be used to improve the mating behavior in males.

### Research Summary

Studies have shown that Maca can reduce enlarged prostate glands in rats. Small-scale clinical trials performed in men have shown that maca extracts can heighten libido and improve semen quality, though no studies have been performed on men with sexual dysfunction or infertility. Maca does not affect sex hormone levels in humans, and has not been shown to act on hormones directly. It has been presumed that maca's hormone-normalizing effects may be due to the root's unique nutritional profile, which provides optimum levels of nutrients utilized by the body's endocrine system. In addition, according to another study, maca has been shown to increase mating behavior in male mice and rats.

Contradictions, Precautions, and Adverse Reactions

Contradictions: No absolute contraindications are known for maca, although reliable information about its use in pregnant or breastfeeding mothers or in young children is not available. There are always a few individuals who will show an allergic reaction or who fall into a group of women or men for whom a pituitary stimulator such as maca is contraindicated in the absence of studies that prove its safety.

Precautions: Cautions should be maintained while using maca in pregnant and lactating women, and in patients suffering from thyroid disorders. Men using maca on a regular basis should undergo periodic PSA tests.

Adverse reactions: Maca is consumed as food for humans and livestock, suggesting any risk from consumption is rather minimal. It is considered safe to eat as any other vegetable food. However, maca does contain glucosinolates, which can cause goitres when high consumption is combined with a diet low in iodine.

Interactions: When taken with diet low in iodine, maca can cause goiters.

Over-dosage: not documented.

Dosage and administration: One should take 3,000-5,000 mg per day of maca.

### Description

### Lutein

Lutein is one of over 600 known naturally occurring carotenoids. Found in green leafy vegetables such as spinach and kale, lutein is employed by organisms as an antioxidant and for blue light absorption. Lutein is present in the plant as fatty-acid ester, with one or two fatty acids bound to the two hydroxyl-groups. Lutein is also found in egg yolks, animal fats, and the retina (lutein predominates elsewhere in the retina except macula lutea). Lutein is a lipophilic molecule and is generally insoluble in water. The presence of the long chromophore of conjugated double bonds (polyene chain) provides the distinctive light-absorbing properties. The polyene chain is susceptible to oxidative degradation by light or heat and is chemically unstable in acids.

Carrots, squash and other orange and yellow fruits and vegetables are sources of lutein. Green leafy vegetables, such as spinach, also contain high amounts of lutein. All are an important part of a healthy diet. Lutein is concentrated in the retinas of your eyes and is necessary for good vision. A diet rich in lutein may lower your risk of developing cataracts and macular degeneration. Lutein may also help prevent or slow down atherosclerosis, the thickening of arteries, which is a major risk for cardiovascular disease.

### Actions and Pharmacology

Actions: Carotenoids lutein is accumulated in the retina, especially in the macula lutea. It is able to absorb blue light, which damages photoreceptors and pigmentary epithelium. Due to its antioxidative properties it can reduce changes in membrane permeability via quenching reactive oxygen species and free radicals.

### Mechanism of Action:

Lutein has been associated with a decreased risk of macular degeneration and cataracts. The physiologic function of lutein in the macular membranes is not known at this time. Referred to as a nonprovitamin A carotenoid, it is not known to have any vitamin A activity. Other possible actions for carotenoids are antioxidant, immunoenhancement, inhibition of mutagenesis and transformation, and inhibition of premalignant lesions. Lutein has been associated with decreased risk of colon cancer and atherosclerosis.

### Pharmacokinetics

Intestinal absorption of carotenoids, including lutein, is facilitated by the formation of bile acid micelles containing carotenoids. The presence of fat in the small intestine stimulates the secretion of bile acids from the gall bladder and improves the absorption of carotenoids by increasing the size and stability of the micelles, thus allowing more carotenoids to be solubilized. Bioavailability of lutein is affected by the dose and presence of other carotenoids such as Beta carotene. The bioavailability of lutein from vegetables is approximately 70%.

### Indications and Use

Carotenodermia is a harmless biological effect of high carotenoid intake. Characterized by a yellowish discoloration of the skin, it results from chronically elevated serum concentrations of carotenes.

### Research Summary

In a study at North Chicago VA Medical Center 90 AMD patients were supplemented daily with an OcuPower supplement capsule containing 10 mg of crystalline FloraGLO lutein, 10 mg lutein plus a mixed antioxidant formula, or placebo for 12 months. The average American ingests one to two mg of lutein daily. Patients ingesting the lutein supplement experienced significant improvements in several objective measurements of visual function including glare recovery, contrast sensitivity, and visual acuity vs. placebo. Patients also experienced a 50% increase in macular pigment density relative to those on placebo.

One study concluded that prospective data from a large cohort of female health professionals indicate that higher intakes of lutein and vitamin E are associated with decreased risk of cataract. The scientists analyzed dietary information from 35,551 female health professionals who enrolled in the Women's Health Study in 1993. The women were then followed for an average of 10 years, and the diets of those who developed cataracts were compared with the diets of those who did not.

According to one article a total of 2,031 women developed cataracts during the study. When the participants were split into five groups based on the amount of lutein and zeaxanthin they consumed, those in the group who consumed the most (about 6,716 micrograms per day) had an 18 percent lower chance of developing cataracts than those who consumed the least (1,177 micrograms per day). The one-fifth who consumed the most vitamin E from food and supplements about 262.4 milligrams per day were 14 percent less likely than the one-fifth who got the least (4.4 milligrams per day).

For coronary heart disease epidemiological data from two studies suggests that lutein may have a protective effect against the progression of early atherosclerosis. High circulating xanthophyll levels indicated reduced carotid artery intima media thickness. In another study, measures of oxidative stress biomarkers was used to determine the relationship between the xanthophylls and risk for heart disease. In the plasma of patients with congestive heart failure, it was found that lutein was significantly lower and malondialdehyde, a product of lipid peroxidation, was significantly higher than in 55 controls.

For stroke among a cohort of male smokers without a history of stroke, a significant inverse relationship was observed between intake of lutein and zeaxanthin and risk for stroke after more than six year follow-up. In plasma, there was an inverse correlation between lutein and malondialdehyde in ischemic stroke patients in comparison, to controls. The findings suggest that lutein may improve clinical outcomes following ischemic stroke.

For gastric cancer, a study suggests a potential adverse association between xanthophylls and gastric cancer, finding that high baseline serum concentrations of lutein and zeaxanthin were directly associated with gastric carcinoma. Several other studies indicate no association between lutein and the development of gastric carcinoma.

For kidney cancer, Yuan et al. found strong inverse associations between the intake of cruciferous and dark green leafy vegetables and cancer risk. A significant inverse association of lutein intake with kidney cancer risk was also observed.

For ovarian cancer, a case controlled study conducted by Bidoli et al. showed that those in the highest quintile of lutein plus zeaxanthin intake had a 40% lower risk of developing ovarian cancer than those in the lowest quintile of intake.

For lung cancer, some studies report a large reduction in the incidence of lung cancer in those with the highest lutein and zeaxanthin intakes; whereas others report no statistically significant relationship between the two.

For breast cancer the associations of xanthophyll intake or serum levels with breast cancer risk in humans has been investigated in many epidemiological studies and the results are not clear. However, studies in human mammary cells and in animal models do support a protective role of xanthophylls against breast cancer.

### Contradictions, Precautions, and Adverse Reactions

### Contradictions:

Lutein should be contraindicted in patients with liver or kidney diseases and women who are pregnant or breast-feeding.

### Precautions:

Due to lack of sufficient medical study, lutein should be used with caution in children, women who are pregnant or breast-feeding, and people with liver or kidney disease.

Adverse reactions: Higher concentrations of plasma lutein may moderately increase cardiovascular disease risk.

### Interactions:

Lutein is not known to interact adversely with any drugs or dietary supplements.

Over-dosage: Not Documented

Dosage and administration: 20-40 mg per day can be considered safe.

### Lycopene

### Description

Lycopene is a red pigment found in fruits and vegetables and is a member of the carotenoid family of chemicals.

Lycopene and beta-carotene are the most abundant carotenoids in the human body. Carotenoids are the pigments that give colour to fruits and vegetables. Many dietary carotenoids are converted to vitamin A in the body, but this is not the case for Lycopene. Lycopene is a powerful antioxidant, which means it protects the body against toxic, free radical chemicals that may contribute to premature ageing and the development of certain diseases, such as the formation of cancers. The intake of tomato-based foods (rich in Lycopene), especially processed tomato products, is associated with a significantly lower risk for prostate cancer.

The risks of breast cancer and cancers of the digestive tract, throat and nose may also be reduced by dietary Lycopene. Lycopene may also help prevent cardiovascular disease by blocking the production of cholesterol in the body. Lycopene may help reduce the damage to the skin caused by ultraviolet light during and after sun exposure. Exposure to certain types of UV radiation can cause damage to DNA (the genetic material of the body) and increase the risks of skin cancer.

### Actions and Pharmacology

Actions: Lycopene is a very efficient antioxidant, which can neutralize oxygen derived free radicals. The oxidative damage caused by these free radicals has been linked to many degenerative diseases such as cardiovascular diseases, premature aging, cancer and cataracts. In many countries it is legally allowed to advertise foods containing tomato lycopene as “containing antioxidants for the maintenance and support of healthy cells”. Lycopene is generally known for its protective action against prostate cancer.

Mechanism of Action: Lycopene has the capacity to prevent free radical damage to cells caused by reactive oxygen species. It is a potent antioxidant in vitro and in human studies, reducing the susceptibility of lymphocyte DNA to oxidative damage, inactivating hydrogen peroxide and nitrogen dioxide, and protecting lymphocytes from nitrogen oxideinduced membrane damage and cell death twice as efficiently as beta-carotene. Evidence is accumulating to suggest other mechanisms of action for lycopene, including modulation of intercellular gap junction communication, an anticancer mechanism. In addition, lycopene at physiological concentrations has been shown to inhibit human cancer cell growth by interfering with growth factor receptor signaling and cell cycle progression, specifically in prostate cancer cells.

### Pharmacokinetics

After ingestion, lycopene is incorporated into lipid micelles in the small intestine. These micelles are formed from dietary fats and bile acids, and help to solubilize the hydrophobic lycopene and allow it to permeate the intestinal mucosal cells by a passive transport mechanism. Little is known about the liver metabolism of lycopene, but like other carotenoids, lycopene is incorporated into chylomicrons and released into the lymphatic system. In blood plasma, lycopene is eventually distributed into the very low and low density lipoprotein fractions. Lycopene is mainly distributed to fatty tissues and organs such as the adrenal glands, liver, and testes.

### Indications and Use

Lycopene is indicated in preventing any conditions which is caused by oxidative damage to any parts of human body. It's used to combat cancer of prostate, stomach, lung, colon and skin. It may also be used in fighting infections caused by bacteria and fungus, and combating complications of diabetes, such as diabetes neuropathies or infection. Lycopene also has role in preventing arteriosclerosis.

### Research Summary

Research showed that Lycopene has a structure similar to that of the well-known antioxidant beta-carotene, but its antioxidant activity is much stronger. Treatment of cells will lycopene protects cells against DNA damage and lipid peroxidation.

Research unveiled that in laboratory conditions lycopene shows antitoxic properties against many toxins such as aflatoxin, cyclosporine and cadmium.

In vitro-studies have shown the anti-cancer properties of lycopene against many cancer cells, including cancer cells of prostate, stomach, lung, colon and skin. There are numerous studies about the effect of lycopene on cancer and prostate cancer in particular.

Studies showed that Lycopene possesses antibacterial and antifungal properties. Lycopene can help to reduce inflammation of the gums and can help to fight infections of Candida albicans.

Diabetes patients may suffer from complications as vascular disease, diabetic neuropathies or infections. Lycopene helps to protect diabetes patients against cardiovascular disease and may improve the immune response, according to studies.

Lycopene inhibits platelet aggregation and reduces the production of foam cells which play an important role in the development of arteriosclerosis, according to studies. Lycopene helps to prevent arteriosclerosis by reducing inflammatory agents in rats increased risk of venous thrombosis.

### Contraindications, Precautions, and Adverse Reactions

Contraindications: Preliminary research suggests lycopene might worsen established prostate cancer by increasing metastasis without having any effect on cancer cell proliferation

Precautions: It should be administered with caution in pregnant and lactating women.

Adverse reactions: Lycopene is non-toxic and is commonly found in the diet, but cases of excessive carotenoid intake have been reported. In a middle aged woman who had prolonged and excessive consumption of tomato juice, her skin and liver were colored orange-yellow and had elevated levels of lycopene in her blood. After three weeks on a lycopene-free diet her skin color returned to normal.

Interactions: Not documented.

Over-dosage: Excessive consumption of lycopene may lead to orange-yellow discoloration of skin and internal organs.

Dosage and administration: Lycopene is advocated orally at the dosage of 6-60 mg per day.

### Noni

### Description

Noni, also known as Indian Mullberry (Morinda citrifolia), is a tropical plant. The leaves, bark, root and fruits of the plant are used as traditional remedy for many diseases. The constituents of Noni include Alkaloids (xeronine); Polysaccharides (glucuronic acid, galactose, arabinose, trisaccharide fatty acid ester); Scopoletin; Terpene; Vitamins & Minerals (magnesium, iron, potassium, selenium, zinc, copper sulphur, ascorbic acid); Antraquinones (damnacanthal); Glycosides (flavonol glycoside, iridoid glycoside, citrifolinoside); amino acids; fatty acids Morindin; and Morindone.

### Actions and Pharmacology

Actions: Noni acts as a potent immune-booster, antioxidant and anti-inflammatory agent. It also acts as an anti-tumor agent.

Mechanism of Action: Noni stimulates and boosts the body's immune system and so enables the body to produce the substance nitric oxide (NO) in the body. This chemical allows the blood vessels to dilate more easily and be more elastic. This means that it acts as a vasodilator (causing vessels to expand) to reduce the high blood pressure. Another phytochemical that has been connected to lowering blood pressure is Scopoletin it has been proven to dilate blood vessels and act as a vasodilator also helping to lower the blood pressure. Serotonin, a neurotransmitter (allows nerve cells to communicate and interact with one another) helps to regulate the expansion- and contraction of blood vessels and the function of platelets (cells that cause blood to coagulate and close a wound). Serotonin has more and other complex roles in regulating blood flow to the brain, heart, and gastrointestinal tract. Morinda citrifolia helps to keep and maintain the serotonin level in blood and thus helps in improving forms of vascular disease. The fruit juice is a big source of (beta) sitosterol, stigmasterol, and campe sterol—the three most nutritionally important phytosterols. Plant sterols, called phytosterols alleviate problems associated with high levels of “bad” cholesterol (LDL). The noni fruit helps in reducing serum cholesterol level and thus prevents further cardiac problems. Ripe fruit contains a concentration of anthraquinones including damnacanthal, which has purgative activity. This accounts for the so called cleansing effect. With sluggish digestion and slow moving bowels, the noni fruit can exert a stimulating effect, which helps to increase peristalsis and cleanse the colon. In studies, noni demonstrated immunomodulatory and antitumor activity. It may be a supplementary agent in cancer treatment. Okadaic acid in the fruit has been known to increase the synthesis of tumor necrosis factor.

### Pharmacokinetics

Noni is advocated orally and is absorbed through the oral route.

### Indications and Use

Noni is used to combat hypertension and cardiac disorders. It also works as a cholesterol-lowering agent and as a purgative. As an anti-oxidant and anti-inflammatory agent, Noni is useful in fighting urinary disorders, muscle and joint pain, and precancerous and cancerous conditions. It may also be used as an immune-booster.

### Research Summary

Studies suggested that Noni has specific role in combating vascular ailments.

Noni can be effective in controlling hypertension, according to research.

Studies have also suggested that Noni has anti-tumor activities and it helps fight precancerous conditions as well as cancers.

Contraindications, Precautions, and Adverse reactions

Contraindications: Patients with renal diseases, diabetes and breathing problems should avoid it.

Precautions: Noni should not be taken empty stomach.

Adverse reactions: It can cause constipation and rashes.

### Interactions

There are no well-known drug interactions with Noni.

Over-dosage: Overdosage can cause constipation.

Dosage and administration: 2-6 ml per day.

### Phosphatidylserine

### Description

Phosphatidyl Serine is a phospholipid that is comprised of the amino acid L-serine and a lipid molecule. Phosphatidylserine is a phospholipid component, usually kept on the inner-leaflet, the cytosolic side, of cell membranes by an enzyme called flippase. When a cell undergoes apoptotic cell death phosphatidylserine is no longer restricted to the cytosolic part of the membrane, but becomes exposed on the surface of the cell. It is present in the cells of all plants and animals, and is derived from the cerebral cortex of cattle, or from egg yolks, soybeans or lecithin (phosphatidylcholine). As a nutritional supplement, phosphatidyl serine is derived from plant sources to eliminate the risk of disease transmission from infected cattle.

Phosphatidyl serine (PS) is present in the cells of every living plant and animal. It is vital to the structure and functioning of cells, and in humans it is vital to proper brain cell function and brain operation.

While its mechanisms of action are unknown, PS is thought to maintain nerve cell integrity, enhance neurotransmitter signal efficiency, enhance cognition, elevate mood, enhance memory, increase the brains absorption of nutrients, and improve nerve cell signal transmission. Phosphatidylserine enables your brain cells to metabolize glucose and to release and bind with neurotransmitters, all of which is important to learning, memory and other cognitive functions.

Phosphatidylserine increases communication between cells in your brain by increasing the number of membrane receptor sites for receiving messages. Phosphatidylserine modulates the fluidity of cell membranes—essential to your brain cells' ability to send and receive chemical communication.

Scientific studies demonstrate that phosphatidylserine restores the brain's supply and output of acetylcholine, the neurotransmitter so important to memory, and so may turn back the clock in an aging brain.

Phosphatidylserine can increase the availability of endogenous (that which is created within your cells) choline for de novo synthesis and release (while similar treatments with phosphatidylcholine had no effect).

Phosphatidylserine also stimulates brain to produce dopamine and this is likely why patients diagnosed with clinical depression have shown marked improvement in their symptoms as a result of taking phosphatidylserine daily. Reduced dopamine levels are also thought to contribute to attention deficit disorder and this natural substance has proven to be an effective therapeutic agent for ADD and ADHD.

Studies examining athletes involved in cycling, weight training and endurance running show that phosphatidylserine can speed recovery, prevent muscle soreness and help athletes to feel their best during the rigors of training.

Phosphatidylserine is important in bone matrix formation, testicular function, beat coordination of the heart, hormone secretion by the adrenal glands and cell repair and removal by the immune system.

Modern science has only begun to understand how important phosphatidylserine is to our bodies. After all, it's present in every type of cell in our body and the membrane proteins that it activates are important in all cells.

### Actions and Pharmacology

### Actions:

Phosphatidylserine is the major fatty substance in the brain. It protects the function of brain cells by stabilizing the membranes of the cells. It is the most abundant phospholipid in the human brain and is important in neuronal membrane functions such as maintenance of the cell's internal environment, signal transduction, secretory vesicle release, cell-to-cell communication, and cell growth regulation. Phosphatidylserine may increase brain function by limiting the deterioration of cell membranes that contribute to brain aging. PS plays a major role in determining the integrity and fluidity of cell membranes. Without sufficient levels of PS, brain cells do not transmit nerve impulses properly.

Phosphatidylserine is a fat-soluble phospholipid that occurs endogenously in humans. Phosphatidylserine is also a component of the mitochondrial membrane, where it might function as a metabolic reservoir for other phospholipids. Although the body is able to synthesize phosphatidylserine through an elaborate series of reactions and substantial energy expenditure, the body obtains most phosphatidylserine from dietary sources. Phosphatidylserine is present in small quantities in most foods.

It is not clear how phosphatidylserine works for dementia such as Alzheimer's disease and age-related memory impairment. However, one theory is that patients with dementia or age-related memory impairment have structural or functional abnormalities in neuronal membranes that cause changes in neurotransmitter functioning. People with cognitive dysfunction often have changes in acetylcholine, norepinephrine, and serotonin levels. Some researchers think the abnormal neuronal function can be attributed to changes in lipid composition of the brain. It is thought that exogenous administration of phosphatidylserine might then normalize brain lipid content and return neuronal function to normal. Phosphatidylserine has been shown to increase acetylcholine, norepinephrine, serotonin, and dopamine levels in animal models and patients with Alzheimer's disease.

In animal models, levels of phosphatidylserine in the brain decline with age. In animal models, phosphatidylserine improves spatial memory and passive avoidance. Phosphatidylserine also appears to minimize age-related neuronal dendrite loss and atrophy of cholinergic neurons. The fatty acid docosahexaenoic acid (DHA), which is readily present in neuronal cells, appears to further promote the accumulation of phosphatidylserine in cell membranes, which in turn prevents apoptotic cell death.

Evidence shows that high levels of procoagulant endothelial particles containing phosphatidylserine are present in patients with acute coronary syndromes. Although these increased levels are hypothesized to contribute to plaque disruption and thrombosis, the exact mechanisms are not yet understood.

There is also interest in phosphatidylserine for decreasing exercise-induced stress. Some preliminary evidence shows that phosphatidylserine might blunt the rise in cortisol and adrenocorticotropin following strenuous training. Very preliminary clinical laboratory research suggests that phosphatidylserine 300 mg per day might improve mood and subjective feelings of stress.

In animal models of multiple sclerosis, phosphatidylserine reduces tremor, spasticity, and urinary incontinence, possibly by suppressing the release of the cytokine tumor necrosis factor.

### Mechanism of Action:

Phosphatidylserine modifies glucose metabolism in the brain, catecholamine and acetylcholine release, NMDA receptor density and function, and muscarinic acetylcholine receptor density, and all of these effects are correlated to the behavioral changes following acute administration.

The primary mechanism of action appears to be an enhancement of cholinergic transmission. Phosphatidylserine administration to old rats restores acetylcholine release to the level of young rats, as well as increasing acetylcholine receptor density. A cholingergic mechanism is also indicated by the reversal of scopolamine-induced amnesia. Phosphatidylserine increases cholinergic function in multiple ways. First, it enhances the activity of Na+, K+-ATPase, which helps maintain membrane potential. Secondly, it increases Ca2+ uptake into K+-depolarized corticol synaptosomes, and this is an important event in neurotransmitter release. Finally, phosphatidylserine affects exocytosis of neurotransmitters by interacting with membrane-binding proteins.

In addition to cholinergic mechanisms, phosphatidylserine may improve memory by increasing the turnover of dopamine and/or norepinephrine (NE) in the brain. In vivo, phosphatidylserine increases turnover of NE in the hypothalamus and dopamine in the striatum. It also increases dopamine release in the limbic area and cerebral cortex of aged animals to normal levels. Chronic phosphatidylserine also affects NMDA receptor function in the forebrain of aged mice, and the blockade of prolonged step-through latency caused by cycloheximide suggests serotonergic mechanisms.

All of this indicates that in addition to specific mechanisms, phosphatidylserine may improve brain function through more non-specific mechanisms. It has been shown that exogenous phospholipids can provide an extra supply for endogenous phospholipid turnover in membranes. Phosphatidylserine mediates a variety of processes related to synaptic plasticity, information storage, and glutamatergic transmission. It also acts as an antioxidant, suppresses cytotoxic factors such as TNF-alpha and nitric oxide, interacts with nerve growth factor (NGF), and increases brain glucose concentration, so the effect on memory could be due to any combination of these factors.

Pharmacokinetics Not well documented.

Indications and use Not well documented.

### Research Summary

A study investigated the effects of 750 mg of soybean-derived phosphatidylserine, administered daily for 10 d, on exercise capacity, oxygen uptake kinetic response, neuroendocrine function, and feeling states during exhaustive intermittent exercise. This is the first study to report improved exercise capacity following phosphatidylserine supplementation. These findings suggest that phosphatidylserine might possess potential ergogenic properties.

Phosphatidylserine, derived from cow brains, has been shown previously to dampen the ACTH and cortisol response to physical stress. Further research investigated the influence of soy lecithin phosphatidylserine supplementation on mood and heart rate when faced with an acute stressor. In this study, we investigated the effects of soy lecithin phosphatidic acid and phosphatidylserine complex (PAS) supplementation on pituitary adrenal reactivity (ACTH, cortisol) and on the psychological response (Spielberger State Anxiety Inventory stress subscale) to a mental and emotional stressor. Four groups of 20 subjects were treated for three weeks with daily dosages of either 400 mg PAS, 600 mg PAS, 800 mg PAS, or placebo before exposure to the Trier Social Stress Test (TSST). Treatment with 400 mg PAS resulted in a pronounced blunting of both serum ACTH and cortisol, and salivary cortisol responses to the TSST, but did not affect heart rate. The effect was not seen with larger doses of PAS. With regard to the psychological response, 400 mg PAS seemed to exert a specific positive effect on emotional responses to the TSST. While the placebo group showed the expected increase in distress after the test, the group treated with 400 mg PAS showed decreased distress. These data provide initial evidence for a selective stress dampening effect of PAS on the pituitary-adrenal axis, suggesting the potential of PAS in the treatment of stress related disorders.

Another research reported the results of a study of the safety of two dosages of soy-phosphatidylserine in elderly. Subjects were 120 elderly of both sexes who fulfilled the more stringent criteria for age-associated memory impairment; some also fulfilled the criteria for age-associated cognitive decline. Subjects were allocated at random to one of the three treatment groups: placebo, 300 or 600 mg S-phosphatidylserine daily. Standard biochemical and hematological safety parameters, blood pressure, heart rate and adverse events were assessed at baseline, after 6 and 12 weeks of treatment. No significant differences were found in any of the outcome variables between the treatment groups after Bonferonni-Holme correction. In conclusion, soy derived phosphatidylserine is a safe nutritional supplement for older persons if taken up to a dosage of 200 mg three times daily.

There have been previous reports that supplements of phosphatidylserine blunted the release of cortisol in response to exercise stress and that it improved mood. The present study extended these observations by considering whether phosphatidylserine supplementation influenced subjective feelings of stress and the change in heart rate when a stressful mental arithmetic task was performed. In young adults, with neuroticism scores above rather than below the median, the taking of 300 mg phosphatidylserine each day for a month was associated with feeling less stressed and having a better mood. The study for the first time reports an improvement in mood following phosphatidylserine supplementation in a sub-group of young healthy adults.

One study assessed whether the efficacy of plant-source derived phosphatidylserine for treatment of age related cognitive decline is consistent with previous (placebo controlled) positive findings with bovine derivative of PS (BC-PS). Eighteen healthy elderly volunteers meeting Age Associated Memory Impairment inclusion and exclusion criteria were treated for 12 weeks with plant-source derived phosphatidylserine (PS) (100 mg×3/day p.o.) and evaluated at base line, after 6 weeks of treatment and at the end of the trial. Fifteen concluded the study. All but two outcome measures elicited a significant drug over time effect. Post-hoc paired t-tests showed that the significant effect was attributable to an improvement from base line to week 6 and that effect was maintained at week 12.

### Contradictions, Precautions, and Adverse Reactions

### Contradictions:

Patients with high blood pressure or heart or blood vessel disease and who are taking are taking medicine or are allergic to any medicine or pregnant women should not take it.

### Precautions:

Before taking Phosphatidylserine, tell your doctor if you are pregnant or breastfeeding

### Adverse Reactions:

Side effects are rare but may include nausea, upset stomach, or trouble falling asleep if Phosphatidylserine is taken before bed.

Interactions: Some drying medications are called anticholinergic drugs. Phosphatidylserine might increase chemicals that can decrease the effects of these drying medications.

Over-dosage: Not Documented

Dosage and administration: 100-200 mg daily.

Uncaria tomentosa

### Description

Uncaria tomentosa (Cat's Claw) is a woody vine found in the tropical jungles of South and Central America, which derives its name from its claw-shaped thorns. It is used as an alternative medicine in the treatment of a variety of ailments.

Cat's claw, an herb found in a limited region of the upper Andes in Peru, is one of several dozen herbs being promoted these days as an effective treatment, even a potential cure, for cancer, AIDS, chronic fatigue syndrome, candida infection, arthritis, and other disorders for which modern medicine is often unsatisfactory. The broad spectrum of action claimed for these herbs is not an impossibility, as the disorders that are said to be treated involve the immune system: there could be a central regulatory mechanism affected by a natural compound that leads to improvements for many patients with various diseases. The suggestion that the herbs produce dramatic effects or are curative rather than merely helpful is more problematic, as clinical evaluations of several such materials have failed to confirm many of the claims that were based on individual case studies. For example, among claimed alternative medicine cancer cures, follow-ups conducted by independent investigators have almost always led to an examination of death certificates.

Cat's claw is a vine native to South America. The bark of this plant has been used in traditional medicine to treat diseases. It is also a very popular immune-enhancing supplement. In vitro studies show that the alkaloids from Cat's claw enhance phagocytosis, display immunomodulatory properties, alleviate inflammation, and possess anti-viral activity. Cat's claw is also thought to have anticancer activities and lab results demonstrated growth inhibitory effects on glioma and neuroblastoma cells as well as promyelocytic leukemia cells. However, no human studies have been conducted to evaluate efficacy. Reported adverse reactions include hypotension and diarrhea. An additive effect with anticoagulants or hypotensives is possible; therefore caution should be exercised.

### Actions and Pharmacology

### Actions:

Cat's claw is a remarkably potent inhibitor of TNFalpha production. The primary mechanism for cat's claw anti-inflammatory actions appears to be immunomodulation via suppression of TNFalpha synthesis. TNF stands for tumor necrosis factor. Cat's claw is an effective antioxidant. Cat's claw extracts exert a direct anti-proliferative activity on MCF7 (a breast cancer cell line). This plant is an effective treatment for osteoarthritis and probably rheumatoid arthritis. The species, U guianensis and U tomentosa are equiactive. They are effective antioxidants, but their anti-inflammatory properties may result from their ability to inhibit TNFalpha and to a lesser extent PGE2 production.

Human volunteers who took cat's claw herb for 8 weeks had improved DNA repair. Cat's claw herb total alkaloids exert a beneficial effect on memory impairment induced by the dysfunction of cholinergic systems in the brain and that the effect of the total alkaloids is partly attributed to the oxindole alkaloids tested.

The oxindole alkaloids are claimed to have immunostimulating properties in vitro, increasing phagocytotic activity and synthesis of WBCs and enhancing T-helper cell function. The major alkaloid, rhynchophylline, is claimed to be anti-hypertensive; it relaxes the endothelial cells of blood vessels, dilates peripheral blood vessels, inhibits sympathetic nervous system activities, and lowers the heart rate and blood cholesterol. The alkaloid mytraphylline has diuretic properties, and hirsutine inhibits urinary bladder cotractions and possesses local anesthetic.

### Mechanism of Action:

The active components of cat's claw are mainly alkaloids, glycosides (triterpenes and procyanidins), and tannins. The oxindole alkaloids of the stem (including the hooks) are the same as those found in the Chinese plant that is far more intensively analyzed. Rhynchophylline, the main alkaloid, has been made into a drug in China for treating hypertension and headache due to vascular constriction. The alkaloids in the root bark of cats' claw are in the same category as rhynchophylline, but are slightly differently. The claim made by some investigators appears to be that these unique alkaloids are responsible for the ability of the plant to treat cancer and to inhibit viral infections. Enhancement of phagocytosis in vitro was reported in 1985 by Wagner, a European researcher who has focused efforts on revealing immune-enhancing actions of natural products (his work with echinacea, eleutherococcus, and the liver-protective herb sylibum is frequently reported in the alternative medicine literature). However, it is not clear that sufficient amounts of such alkaloids are consumed so that one would obtain this effect nor how strong the effect might be. Based on his research experience, Wagner believes that polysaccharides, terpenoids, alkaloids, and polyphenolic compounds from plants have immunostimulating activity.

Pharmacokinetics: Insufficiently documented.

### Research Summary

To evaluate safety and clinical efficacy of a plant extract from the pentacyclic chemotype of Uncaria tomentosa (cat's claw) in patients with active rheumatoid arthritis (RA). Forty patients undergoing sulfasalazine or hydroxychloroquine treatment were enrolled in a randomized 52 week, 2 phase study. During the first phase (24 weeks, double blind, placebo controlled), patients were treated with cats claw extract or placebo. In the second phase (28 weeks) all patients received the plant extract. Twenty-four weeks of treatment with the cats claw extract resulted in a reduction of the number of painful joints compared to placebo (by 53% vs 24%). Patients receiving the cat's claw extract only during the second phase experienced a reduction in the number of painful and swollen joints and the Ritchie Index compared to the values after 24 weeks of placebo. Only minor side effects were observed. This small preliminary study demonstrates relative safety and modest benefit to the tender joint count of a highly purified extract from the pentacyclic chemotype of cat's claw in patients with active RA taking sulfasalazine or hydroxychloroquine.

In this study, the effects of two cat's claw extracts and two mixtures of tetracyclic and pentacyclic oxindole alkaloids were investigated in freshly isolated human peripheral blood mononuclear cells (PBMC) stimulated with the mitogens phytohaemagglutinin (PHA) and concanavalin A (Con A) in vitro. These data demonstrate that cat's claw extracts and mixtures of alkaloids modulate the immunobiochemical pathways induced by interferon-gamma. The findings imply a potential application of the cat's claw extracts as immunoregulators and would be in line with observations in patients using these extracts.

The Uncaria tomentosa water extracts (cat's claw, C-Med-100) have been shown to enhance DNA repair, mitogenic response and leukocyte recovery after chemotherapy-induced DNA damage in vivo. In this study, the effect of cat's claw C-Med-100 supplement was evaluated in a human volunteer study. Twelve apparently healthy adults working in the same environment were randomly assigned into 3 groups with age and gender matched. One group was daily supplemented with a 250 mg tablet containing an aqueous extract of cat's claw, and another group with a 350 mg tablet, for 8 consecutive weeks. DNA repair after induction of DNA damage by a standard dose of hydrogen peroxide was measured 3 times before supplement and 3 times after the supplement for the last 3 weeks of the 8 week-supplement period. There were no drug-related toxic responses to cat's claw supplement when judged in terms of clinical symptoms, serum clinical chemistry, whole blood analysis and leukocyte differential counts. There was a statistically significant decrease of DNA damage and a concomitant increase of DNA repair in the supplement groups (250 and 350 mg/day) when compared with non-supplemented controls (p<0.05). There was also an increased tendency of PHA induced lymphocyte proliferation in the treatment groups. Taken together, this trial has confirmed the earlier results obtained in the rat model when estimating DNA repair enhancement by cat's claw.

Decoctions prepared from the bark of Uncaria tomentosa (cat's claw) are widely used in the traditional Peruvian medicine for the treatment of several diseases, in particular as a potent anti-inflammatory agent. Therefore, the main purpose of this study was to determine if the well-known anti-inflammatory activity of cat's claw decoction was related with its reactivity with the oxidant species generated in the inflammatory process and to establish a relationship between such antioxidant ability and its phenolic composition. We observed that the cat's claw decoction prepared according to the traditional Peruvian medicine presented a potent radical scavenger activity, as suggested by its high capacity to reduce the free radical diphenylpicrylhydrazyl, and by its reaction with superoxide anion, peroxyl and hydroxyl radicals as well as with the oxidant species, hydrogen peroxide and hypochlorous acid. Cat's claw also protected membrane lipids against peroxidation induced by the iron/ascorbate system, as evaluated by the formation of thiobarbituric acid-reactive substances (TBARs). The cat's claw decoction phenolic profile was established by chromatographic analysis (HPLC/DAD and TLC) revealing essentially the presence of proanthocyanidins (oligomeric procyanidins) and phenolic acids, mainly caffeic acid. Thus, our results provide evidence for an antioxidant mechanism underlying the anti-inflammatory activity of cat's claw and support some of the biological effects of proanthocyanidins, more exactly its antioxidant and radical scavenging activities.

In the traditional Peruvian medicine, hot aqueous extracts of cat's claw have been used for the treatment of a wide range of health problems, particularly digestive complaints and arthritis. Some of the beneficial effects observed in patients suggest an immunomodulatory capacity of cat's claw extracts. In this study, the effects of two extracts and two mixtures of tetracyclic and pentacyclic oxindole alkaloids of cat's claw were investigated in freshly isolated human peripheral blood mononuclear cells (PBMC) stimulated with the mitogens phytohaemagglutinin (PHA) and concanavalin A (Con A) in vitro. These data demonstrate that cat's claw extracts and mixtures of alkaloids modulate the immunobiochemical pathways induced by interferon-gamma. The findings imply a potential application of cat's claw extracts as immunoregulators and would be in line with observations in patients using these extracts.

Previous reports have demonstrated that extracts of the plant Uncaria tomentosa inhibit tumor cell proliferation and inflammatory responses. We have confirmed that cat's claw C-Med 100, a hot water extract of this plant, inhibits tumor cell proliferation albeit with variable efficiency. We extend these findings by showing that this extract also inhibits proliferation of normal mouse T and B lymphocytes and that the inhibition is not caused by toxicity or by induction of apoptosis. Further, the extract did not interfere with IL-2 production nor IL-2 receptor signaling. Since there was no discrete cell cycle block in cat's claw C-Med 100-treated cells, we propose that retarded cell cycle progression caused the inhibition of proliferation. Collectively, these data suggested interference with a common pathway controlling cell growth and cell cycle progression. Indeed, we provide direct evidence that cat's claw C-Med 100 inhibits nuclear factor kappa B (NF-kappa B) activity and propose that this at least partially causes the inhibition of proliferation.

Cat's claw is an herbal medicine from the Amazon that is used widely to treat inflammatory disorders. The purpose of this study was to characterize the antioxidative and antiinflammatory properties of cat's claw, Uncaria tomentosa and Uncaria guianensis. These results indicate that while both species of cat's claw provide effective antioxidant and anti-inflammatory activities, U. guianensis is more potent. In conclusion, the presence of oxindole or pentacyclic alkaloids did not influence the antioxidant and anti-inflammatory properties of cat's claw.

Uncaria tomentosa, also known as “Una de gato, cat's claw”, is a Rubiaceae species widely used in South-American folk medicine for the treatment of cancer, arthritis, gastritis and epidemic diseases. Extracts of the plant have been shown to possess cytostatic and anti-inflammatory activity as well as mutagenic and antimutagenic properties. However, to date no studies have been carried out to verify the direct antitumor activity of the cat's claw extracts. The present study investigates the effects of some extracts and their chromatographic fractions from the bark of cat's claw on the growth of a human breast cancer cell line (MCF7). Our data indicated that, in addition to the antimutagenic activity, cat's claw extracts and fractions exert a direct antiproliferative activity on MCF7.

Cat's claw (Uncaria tomentosa) is a medicinal plant from the Amazon River basin that is widely used for inflammatory disorders and was previously described as an inhibitor of NF-kappaB. Cat's claw was prepared as a decoction (water extraction) of micropulverized bark. Cat's claw suppressed TNFalpha production by approximately 65-85% but at concentrations considerably lower than its antioxidant activity. In conclusion, cat's claw is an effective antioxidant, but perhaps more importantly a remarkably potent inhibitor of TNFalpha production. The primary mechanism for cat's claw anti-inflammatory actions appears to be immunomodulation via suppression of TNFalpha synthesis.

Contradictions, Precautions, and adverse reactions

Contradictions: Cat's claw has been clinically documented with immunostimulant effects and is contraindicated before or following any organ or bone marrow transplant or skin graft. Cat's claw has chemicals that can reduce platelet aggregation and thin the blood.

Precautions: Although the data is insufficient, its use should be avoided in women who are pregnant or who want to become pregnant. Use cautiously in patients with renal dysfunction because cat's claw may be nephrotoxic.

Adverse reactions: Orally, cat's claw can cause headache, dizziness, and vomiting. May cause diarrhea and lower blood pressure.

Interactions: Based upon in vivo rat studies, cat's claw may protect against gastrointestinal damage associated with nonsteroidal anti-inflammatory drugs (NSAIDs) such as ibuprofen. Cat's claw may potentiate coumadin and blood-thinning drugs.

Over-dosage: Not Documented

Dosage and administration: 500 mg-1 g per day.

### Vinpocetine

### Description

Vinpocetine is a semisynthetic derivative alkaloid of vincamine an extract from the periwinkle (plant) Vinca minor.

Vinpocetine is reported to have cerebral blood-flow enhancing and neuroprotective effects, and is used as a drug in Eastern Europe for the treatment of cerebrovascular disorders and age-related memory impairment.

Vinpocetine is widely marketed as a supplement for vasodilation and as a nootropic for the improvement of memory. In other words, Vinpocetine may help support brain functions such as concentration and memory by activating cerebral metabolism. There exists anecdotal report of uncomfortable adverse reactions to vinpocetine in a small subset of users. A low initial dosage is often recommended.

Experiments with this periwinkle extract indicate that it can dilate blood vessels, enhance circulation in the brain, improve oxygen utilization, make red blood cells more pliable, and inhibit aggregation of platelets. Vinpocetine even has antioxidant properties. Levels peak in the bloodstream within an hour and a half after ingestion. Vinpocetine easily crosses the blood-brain barrier.

### Actions and Pharmacology

### Actions:

Vinpocetine exerts a brain neuroprotective effect by a combined action on cerebral circulation, brain metabolism, and rheological properties of the blood. Kiss and Karpati (1996) summarized the pharmacological studies on vinpocetine. Early experiments showed an improvement of the cerebral circulation and oxygen utilization without changes in systemic circulation, cerebral protection in conditions of hypoxia/ischaemia, cognition-enhancing and anticonvulsant activity, and improvement of rheological properties of the blood. Later studies confirmed the above effects and clearly demonstrated a direct neuroprotective action at a cellular level.

### Mechanism of Action:

Vinpocetine has been shown to selectively inhibit voltage-sensitive Na+ channels, resulting in a dose-dependent decrease in evoked extracellular Ca+ ions in striatal nerve endings. The Na+ channel inhibiting properties of vinpocetine are thought to contribute to a general neuroprotective effect through blockade of excitotoxicity and attenuation of neuronal damage induced by cerebral ischemia/reperfusion.

Vinpocetine is also a phosphodiesterase (PDE) type-1 inhibitor, (with an IC₅₀ of approximately 10⁻⁵ M.) leading to increases in intracellular levels of cyclic guanosine 3′5′-monophosphate (cGMP), an action that has been attributed to the vasorelaxant effects of vinpocetine on cerebral smooth muscle tissue.

Increases in neuronal levels of DOPAC, a metabolic breakdown product of dopamine, have been shown to occur in striatal isolated nerve endings as a result of exposure to vinpocetine. Such an effect is consistent with the biogenic pharmacology of reserpine, a structural relative of vinpocetine, which depletes catecholamine levels and may cause depression as a side-effect of the cardiovascular and anti-psychotic effects.

Pharmacokinetics: Vinpocetine is absorbed from the small intestine, from whence it is transported to the liver via the portal circulation. From the liver via the systemic circulation, it is distributed to various tissues in the body, including the brain. Absorption of vinpocetine is significantly higher when given with food and can be up to about 60% of an ingested dose. On an empty stomach, absorption of an ingested dose can be as low as 7%. Peak plasma levels are obtained one to one and a half hours after ingestion. Extensive metabolism to the inactive apovincaminic acid occurs in the liver. Only small amounts of unmetabolized vinpocetine are excreted in the urine, the major route of excretion of apovincaminic acid. Most of a dose is excreted within 24 hours as this metabolite. The elimination half-life of vinpocetine following ingestion is one to two hours.

Indications and use: The primary claim made for vinpocetine is that it decreases fatality and dependency in ischemic stroke. Research results are mixed. Vinpocetine has not been helpful in Alzheimer's disease, but there is some suggestion that it might help some with other dementias and cerebral dysfunction. Very preliminary research additionally suggests that vinpocetine may help protect the eye and ear from injuries caused by trauma (and, in the case of the eye, from infection) and that it might be gastroprotective, ameliorate symptoms of motion sickness and help prevent atherosclerosis.

### Research Summary

There are more than 100 clinical studies on Vinpocetine performed on over 30,000 patients proving its safety and effectiveness.

Researchers at the University of Surrey in Guildford, England administered vinpocetine to patients suffering from mild to moderate dementia (Hindmarch 1991). Two hundred and three patients included in a placebo-controlled, randomized double-blind trial received every day for sixteen weeks either 10 mg doses of vinpocetine three times a day, 20 mg doses of vinpocetine three times a day, or placebo three times a day. There were no clinically relevant side effects reported. Statistically significant cognitive improvements were found in favor of active treatment groups compared to placebo. The patients on 10 mg performed slightly better than those on 20 mg.

In a double blind clinical trial, vinpocetine was shown to offer significant improvement in elderly patients with chronic cerebral dysfunction. Forty-two patients received 10 mg vinpocetine three times a day for thirty days, then 5 mg three times a day for sixty days. Matching placebo tablets were given to another forty patients for the ninety-day trial period. Patients on vinpocetine scored consistently better in all cognitive evaluations. No serious side effects were reported.

Forty patients aged 40 to 65 years who had non-exudative forms of age-related macular degeneration (AMD), including 20 patients with degeneration of the retinal pigment epithelium (RPE), 15 with retinal drusen, and 5 with RPE atrophy were examined. All the patients were divided into 2 groups. Group 1 comprised 20 patients receiving, in addition to conventional therapy, cavinton forte (1 tablet contains 10 mg of vinpocetine). Group 2 (control) included 20 patients receiving conventional therapy (antioxidants, peptide bioregulators, lutein containing agents). Medical treatment was performed during 2 months. After a course of cavinton therapy, patients with AMD were observed to have better visual acuity, improved retinal function, and increased a- and b-wave amplitudes on a macular electroretinogram. There was improvement of ocular blood flow values, which is indicative of better uveal blood supply.

Twelve healthy female volunteers received pre-treatments with vinpocetine 40 mg three times a day or placebo for two days according to a randomized, double-blind crossover design. On the third day of treatment and one hour following morning dosage, subjects completed a battery of psychological tests. Memory was significantly improved following treatment with vinpocetine when compared to placebo.

A double-blind, prospective, randomized, placebo-controlled clinical trial was carried out to test the acute and long-term hemodynamical and beneficial cognitive effects of the vasoactive agent vinpocetine on patients suffering from multiple cerebral infarcts by means of functional transcranial Doppler examinations and by neuropsychological tests. Twenty-six patients (17 men, 9 women) with multiple cerebral infarctions, aged between 50 and 83 years were examined, 14 of whom received vinpocetine and 12 placebo. The functional transcranial Doppler included breath-holding tests, finger movement, word fluency, and picture-discrimination tasks. Twenty-five patients were assessed by neuropsychological battery. No serious side effect was found in the vinpocetine group. The flow velocities were significantly lower in the acute phase after breath holding in the vinpocetine group than in the placebo group. Three months later, the vinpocetine patients did not show any significant worsening in digit span backward test, while the placebo group did. No other significant differences in the neuropsychological test could be detected between the treatment and the placebo groups. Longer lasting and higher dosage of vinpocetine therapy is suggested to prove its potential effect.

The effect of vinpocetine on infarct volume (obtained by 2,3,5-triphenyltetrazolium-chloride staining) was studied in permanent middle cerebral artery occlusion in rats. Vinpocetine treatment significantly decreased infarct volume by 42% compared to control, which was better than the effect of nimodipine (17%) or MK-801 (18%). These results together with former literature data indicate that apovincaminic acid derivatives possessing strong neuroprotective potential may play an important role in the therapy of ischemic stroke.

The authors wished to investigate the kinetics and distribution of vinpocetine in the brain and body after oral administration with PET in order to prove, that PET is useful in the non-invasive in vivo determination of these parameters. Vinpocetine was labelled with carbon-11 and the radioactivity was measured by PET in the stomach, liver, brain, colon and kidneys in healthy male volunteers. The radioactivity in the blood and urine was also determined. After oral administration, [11C] vinpocetine appeared immediately in the stomach and within minutes in the liver and the blood. In the blood the level of radioactivity continuously increased until the end of the measurement period, whereas the fraction of the unchanged mother compound decreased. Radioactivity uptake and distribution in the brain were demonstrable from the tenth minute after the oral administration of the labelled drug (average maximum uptake: 0.7% of the administered total dose). Brain distribution was heterogeneous (with preferences in the thalamus, basal ganglia and occipital cortex), similar to the distribution previously reported by the authors after intravenous administration. Vinpocetine, administered orally to human volunteers, readily entered the bloodstream from the stomach and the gastrointestinal tract and thereafter passed the blood-brain barrier and entered the brain. Radioactivity from [11C]vinpocetine was also demonstrated in the kidneys and in urine. The study demonstrates that PET might be a useful, direct and non-invasive tool to study the distribution and pharmacokinetics of orally administered labeled drugs, such as vinpocetine, active in the central nervous system in the living human body.

In one study it was examined whether vinpocetine can act as an antioxidant and prevent the formation of ROS and lipid peroxidation in rat brain synaptosomes. After ascorbate/Fe2+ treatment a significant increase in oxygen consumption (about 5-fold) and thiobarbituric acid reactive substances (TBARS) formation (about 7-fold) occurred as compared to control conditions. Vinpocetine inhibited the ascorbate/Fe2+ stimulated consumption of oxygen and TBARS accumulation, an indicator of lipid peroxidation, in a concentration-dependent manner. The ROS formation was also prevented by vinpocetine. Oxidative stress increased significantly the fluorescence of the probes 2′,7′-dichlorodihydrofluorescein (DCFH2-DA) (about 6-fold) and dihydrorhodamine (DHR) 123 (about 10-fold), which is indicative of intrasynaptosomal ROS generation. Vinpocetine at 100 microM concentration decreased the fluorescence of DCFH2-DA and DHR 123 by about 50% and 83%, respectively. We conclude that the antioxidant effect of vinpocetine might contribute to the protective role exerted by the drug in reducing neuronal damage in pathological situations.

Contradictions, Precautions, and adverse reactions

### Contradictions:

Vinpocetine is contraindicated in patients with low blood pressure, constipations, seizure disorders, and liver problems. It should not be taken if pregnant or breastfeeding.

### Precautions:

Vinpocetine has been implicated in one case to induce agranulocytosis, a condition in which granulocytes, are markedly decreased. Some people have anecdotally noted that their continued use of vinpocetine reduces immune function.

### Adverse Reactions:

Side effects of vinpocetine may include indigestion, nausea, dizziness, anxiety, facial flushing, insomnia, headache, drowsiness and dry mouth. Vinpocetine may also cause a temporary drop in blood pressure. Some patients had a passing sensation of warmth after injection of the drug.

Interactions: Because vinpocetine decreases platelet aggregation, caution is warranted in patients receiving blood thinning agents.

Over-dosage: Overdosage can cause nausea, dizziness, anxiety.

Dosage and administration: It is recommended that first-time users ingest only 2-5 mg of vinpocetine with meals to make sure they are not hypersensitive to it. Users may then increase the dosage to 10-40 mg a day (which may, although very rarely, cause some light side-effects).

Bacopa monnieri

### Description

The leaves of this plant are succulent and relatively thick. Leaves are oblanceolate and are arranged oppositely on the stem. The flowers are small and white, with four or five petals. Its ability to grow in water makes it a popular aquarium plant. It can even grow in slightly brackish conditions. Propagation is often achieved through cuttings.

Bacopa is a great neurotonic, immuno-modulator, adaptogen, tranquilizing, memory and learning enhancing, cerebral activator, anti-ulcer, antispasmodic, anti-asthmatic ayurvedic herb. Other benefits are anti-allergic, free radicals scavenging effect and as herbal supplement in epilepsy, anxiety and depression.

It has beneficial actions on brain, memory, mental deficiency, Alzheimer's disease, learning skills, anxiety, depression, stress, epilepsy and ADHD children.

### Actions and Pharmacology

### Actions:

Bacopa helps in repair of damaged neurons by enhancing kinase activity, neuronal synthesis and restoration of synaptic activity and ultimately nerve impulse transmission, thus helps in alzheimers and other degenerative disorders of brain. It calms the mind and promotes relaxation thus decreases anxiety and restlessness. It also increases protein synthesis and activity in the brain cells. It also acts as a blood cleanser that stimulates hair and nails growth. In use it has proven useful for epilepsy, improving memory, increasing concentration and for relieving anxiety. In natural medicine Brahmi also plays a major role as a diuretic, tranquilizer, depression treatment, asthma treatment and insanity treatment. It is also used in children for the treatment of ADD or ADHD.

Brahmi is extremely helpful in treating the anxiety disorders. It regulates the blood in the body and eradicates any kind of deposition of the plaque that disrupts the blood flow through the arteries. It also helps in relaxing the spasm that is the main reason for causing anxiety conditions. It also relaxes muscles in the brain therefore helping in releasing the toxins that primarily causes condition of anxiety. Brahmi has Antioxidant, Cardiotonic and Anticancer properties.

### Mechanism of Action:

Two chemicals in bacopa, bacosides A and B, improve the transmission of impulses between nerve cells in brain. The neurobiological effects of these isolated molecules were found to increase protein kinase activity and new protein synthesis, specifically in cells in region of the brain associated with long-term memory. Bacopa also increases your level of serotonin, a brain chemical known to promote relaxation. The herb's ability to boost brain function while reducing anxiety may explain why it helps treat ADHD.

Based on animal study results, bacosides appear to have antioxidant activity in the hippocampus, frontal cortex, and striatum. Animal research has shown Bacopa extracts modulate the expression of certain enzymes involved in generation and scavenging of reactive oxygen species in the brain. In vitro research has shown Bacopa exerts a protective effect against DNA damage in astrocytes and human fibroblasts. In animals Bacopa has a relaxant effect on pulmonary arteries, aorta, trachea, and ileal and bronchial tissue, possibly mediated by inhibition of calcium-ion influx into cell membranes. Bacopa appears to stabilize mast cells in vitro, and possesses anti-inflammatory activity via inhibition of prostaglandin synthesis and lysosomal membrane stabilization. In vitro research suggests an anticancer effect for Bacopa extracts, possibly due to inhibition of DNA replication in cancer cell lines.

### Pharmacokinetics

Compounds responsible for the pharmacological effects of Bacopa include alkaloids, saponins, and sterols. Many active constituents—the alkaloids Brahmine and herpestine, saponins d-mannitol and hersaponin, acid A, and monnierin—were isolated in India over 40 years ago. Other active constituents have since been identified, including betulic acid, stigmastarol, beta-sitosterol, as well as numerous bacosides and bacopasaponins. The constituents responsible for Bacopa's cognitive effects are bacosides A and B.

Indications and use: Not documented.

### Research Summary

In a double blind randomized placebo controlled research study in Australia, at University of Wollongong, this ayurvedic botanical was found to be effective in tests for retention of new information.

In another similar study mentioned in Neuropsychopharmacology, its effects were documented for several weeks and various memory functions were tested with levels of anxiety. The study revealed the same—B. monnieri decreases the rate of forgetting of newly acquired information, verbal learning rate and memory consolidation.

In yet another study, the chronic (3 weeks to 12 weeks) administration of B. monnieri showed significant improvement in speed of visual information processing by IT task, learning rate and memory consolidation as compared to placebo. There was improvement in higher order cognitive functions that depend on memory, learning and environmental factors.

However the single dose of this medicinal herb doesn't show any improvement in cognitive or memory functions. The dosage in the above two studies was 300 mg of B. monnieri extracts daily.

B. monnieri was tested on men with mild to moderate mental deficiency. 172 persons received B. monnieri 500 mg of extract thrice a day while 114 persons received placebo for one year. At the end of study, there was improvement in concentration ability, memory span, and overall mental performance in individuals taking the extract as compared with placebo group.

There was improvement in the performance of school children with poor educational performance.

Loss of cholinergic activity in hippocampus was the primary cause of Alzheimer's disease. B. monnieri showed important antioxidant activity in many brain parts like hippocampus, striatum and frontal cortex. Further studies showed its protective effect against DNA damage in astrocytes and fibroblast cells. All this suggest its important role in Alzheimer's and at least it could be useful in checking the progression of this disease to some extent.

Despite its mention as anti-epilepsy role it was found to exert this effect only on very high doses over long periods. The dose near LD50 showed effect against seizures. Research in India found hersaponin to exert some anticonvulsant effect. So it could better be used as an adjuvant in treatment of Epilepsy.

Anti-epilepsy drugs as Phenyloin have some side effects as cognitive impairment. Simultaneous administration of this botanical with phenyloin improved acquisition and retention of memory. B. monnieri extracts have corrective effect on phenyloin induced cognitive deficit.

Researchers in a study on rats concluded as “B. monnieri helps in coping with combined hypoxic, hypothermic and immobilization stress that could lead to onslaught of free radicals.”

Research on rats as models of clinical anxiety showed the anxiolytic activity of its extracts with 25 percent bacosides as comparable to Lorazepam. Plus there were no side effects of Lorazepam, like amnesia. Rather there was memory enhancing effect.

Another one month study on diagnosed anxiety neurosis patients, with syrup of this medicinal herb equivalent to 12 gm of crude powder, found significant reduction in anxiety symptoms, level of disability and fatigue. There was additional increase in immediate memory, decreased respiratory rate and decreased BP.

The B. monnieri extract in the dose of 20 to 40 mg per kg was given once daily for five days and it was found comparable to standard anti-depressant drug Imipramine in anti-depressant activity in rodent animals.

Epilepsy patients who are taking Barbiturates can benefit from B. monnieri. It may potentiate the effect of Barbiturates so they are needed in lower dosage. Plus there would be relief from other side effects like behavioral abnormalities, diminution of intelligence, impairment of learning and memory, hyperactivity in children and mental confusion in older patients.

Another double blind study at BRD Medical College, at Gorakhpur, India, on children with ADHD (Attention Deficit Disorder) showed benefit after 12 weeks of B. monnieri use in sentence repetition, logical memory and paired associated learning tasks. The children were given the test four weeks after the B. monnieri had been withdrawn and it affirms its lasting effect.

Researchers believe that, among its other mechanisms, Bacopa meditates the GABAergic system. Gamma-aminobutyric acid is an inhibitory neurotransmitter that has been shown to possess anticonvulsive, antinociceptive (prevention of pain due to hypersensitive nerve endings), locomotor, and sedative effects.

Contradictions, Precautions, and adverse reactions

Contradictions: None Known.

Precautions: No precautions as such.

Adverse reactions: Orally, brahmi can cause nausea, dry mouth, and fatigue.

Interactions: None known

Over-dosage: Not Documented

Dosage and administration: Multiple time dosage will help. One time dosage will not help.

### Description

Dragon's blood is a bright red resin that is obtained from different species of a number of distinct plant genera for example: Croton. It has a long history of use for both the bark and the resin. An early reference dating back to the 1600s notes that Spanish explorer P. Bernabe Cobo found the sap was being used by indigenous tribes throughout Peru and Ecuador. C. lechleri resin and bark are used in traditional medicine in South America. They used it internally and externally to stop bleeding, help heal wounds, and treat intestinal problems. Studies regarding this plant date back to the late 1970s.

### Actions and Pharmacology

### Actions:

Dragon's blood has been used for its antiviral and wound-healing effects. Taspine, a component of dragon's blood, has been documented to have anti-inflammatory and wound-healing actions. Taspine and a proanthocyanidin component also have been shown to have antiviral activities. Animal and laboratory tests have shown some promise for the use of dragon's blood for these medicinal effects. To date, there are no human clinical studies verifying these effects.

### Mechanism of Action:

Not much research has been done on mechanism of action.

Pharmacokinetics: Not much research has been done on pharmacokinetics.

Indications and use Dragon's Blood should be ground into a fine powder for both external and internal applications.

### Research Summary

Dragon's blood also plays a role in GI health. Practitioners are reporting it beneficial for stomach ulcers, ulcerative colitis, and Crohn's disease when taken internally. Preparations made from dragon's blood are found in several pharmaceutical products, some of them patented. A patent describing use of the proanthocyanidin polymer from croton species (SP-303) as an antidiarrheal was issued to Shaman Pharmaceuticals, Inc. USA. A clinical trial of the principal ingredient (SP-303) in patients with HIV-associated diarrhea demonstrated beneficial effects. This important “rainforest resource” has several uses that have been validated by several studies.

The wound-healing action of Dragon's blood resin was first related to the taspine alkaloid in 1989. Several later studies also concentrated on the wound-healing and antitumorous properties of taspine. The lignan dimethylcedrusine was isolated by scientists in 1993 and was shown to play a central role in sangre de grado's effective wound-healing action. This Belgian study revealed that the crude resin stimulated contraction of wounds, helped in the formation of a crust/scab at the wound site, regenerated skin more rapidly, and assisted in the formation of new collagen. This was the study to which Dr. Duke referred in documenting that the crude resin was found to be four times more effective at wound healing and collagen formation than its isolated chemicals (and healed wounds 10-20 times faster than using nothing at all).

The Belgian scientists also determined that taspine was active against herpes virus in this study. In 1994 other phytochemicals were found, including phenolic compounds, proanthocyanadins, and diterpenes, which showed potent antibacterial activity (against E. coli and Bacillus subtilis) as well as wound-healing properties.

Another study documented Dragon's blood antioxidant effects and researchers in Canada documented its antifungal properties. Another important traditional use of the sap was verified by clinical research in a 2000 study designed to evaluate its gastrointestinal effects. Researchers concluded that “Dragon's blood is a potent, cost-effective treatment for gastrointestinal ulcers and distress via antimicrobial, anti-inflammatory, and sensory afferent-dependent actions.” In 2002, these same researchers reported that Dragon's blood evidenced an in vitro effect against stomach cancer and colon cancer cells as well. In 2003 Italian researchers reported that the resin inhibited the growth of a human myelogenous leukemia cell line and also prevented cells from mutating in test tube studies.

Extracts of Dragon's blood have demonstrated antiviral activity against influenza, parainfluenza, herpes simplex viruses I and II, and hepatitis A and B. The antiviral and anti-diarrhea properties of Dragon's blood have come to the attention of the pharmaceutical industry over the last 10 years. A U.S.-based pharmaceutical company has filed patents on three pharmaceutical preparations that contain antiviral constituents and novel chemicals (a group of plant flavonoids they've named SP-303), extracted from the bark and resin of Dragon's blood. Their patented drugs include an oral product for the treatment of respiratory viral infections, a topical antiviral product for the treatment of herpes, and an oral product for the treatment of persistent diarrhea. These products have been the subject of various human clinical trials. Although the immunomodulating effects of Dragon's blood have not been the subject of targeted research yet, some researchers believe that the anti-inflammatory, antimicrobial, and antioxidant activities may provide nonspecific immune enhancement effects as well.

More recently, several scientific tests have been conducted on a proprietary Dragon's blood product (made into a skin balm) which was also based on traditional uses. They reported that in pest control workers, a Dragon's blood balm was preferred over placebo, for the relief of itching, pain, discomfort, swelling, and redness in response to wasps, fire ants, mosquitoes, bees, cuts, abrasions, and allergic plant reactions (poison ivy and others). Subjects reported relief within minutes, and that it provided pain relief and alleviated symptoms (itching and swelling) for up to six hours. These reported effects in humans as well as several other tests they conducted in animals and in vitro models of inflammation led them to conclude that Dragon's blood prevents pain sensation by blocking the activation of nerve fibers that relay pain signals to the brain (therefore functioning as a broad-acting pain killer) as well as blocks the tissue response to a chemical released by nerves that promotes inflammation.

Contradictions, Precautions, and adverse reactions

### Contradictions:

Contraindicated in cases without blood stasis, and during pregnancy.

### Precautions:

Information regarding safety and efficacy in pregnancy and lactation is lacking.

### Adverse Reactions:

There have been no major toxicities reported with the use of dragon's blood.

### Interactions:

None well documented.

Over-dosage: Not Documented

Dosage and administration: 125 to 500 mg daily.

### Guaranine

### Description

Guarana seed is a potent South American energy food with numerous health benefits. Guarana (from the Portuguese guaraná), Paullinia cupana is a climbing plant in the maple family, Sapindaceae, native to the Amazon basin and especially common in Brazil. For thousands of years, native Amazonians have used the seed extract of the guarana berry to help maintain stamina and physical endurance. Guarana features large leaves and clusters of flowers, and is best known for its fruit, which is about the size of a coffee bean. As a dietary supplement, guarana is an effective energy booster: it contains about twice the caffeine found in coffee beans (about 2-4.5% caffeine in guarana seeds compared to 1-2% for coffee beans). Guaranine is defined as only the caffeine chemical in guarana, it is identical to the caffeine chemical derived from other sources, but more powerful, for example coffee, tea, and maté. Guaranine, theine, and mateine are all synonyms for caffeine when the definitions of those words include none of the properties and chemicals of their host plants except the chemical caffeine. Natural sources of caffeine contain widely varying mixtures of xanthine alkaloids other than caffeine, including the cardiac stimulants theophylline and theobromine and other substances such as polyphenols which can form insoluble complexes with caffeine.

### Actions and Pharmacology

### Actions:

The active compound is guaranine, a member of the caffeine family. But unlike regular caffeine, it's full of healthy fatty acids. The good fat gives guaranine a slow release. Its effect will gradually increase over a period of hours and It doesn't pick up and go down like quick release caffeine.

### Mechanism of Action

It works its magic by releasing acetylcholine in the brain. Acetyl-choline is a neurotransmitter. Boosting the level of choline in your blood has a powerful effect on homocysteine and C-reactive protein (CRP).

### Pharmacokinetics

Like caffeine, guarana works by stimulating the adrenal glands to release the hormones epinephrine, norepinephrine, and dopamine, which in turn enhance fat loss, energy, and endurance as well as mental clarity. Contrary to popular belief, these effects can be obtained seemingly without the often-proclaimed negative side effects. Nevertheless, guarana does have dehydrating effects, so increasing water intake is very important with use of this herb.

### Indications and Use

It temporarily gives relief from headaches. It maintains stamina and physical endurance.

### Research Summary

Guarana has been classified as a tonic and a coolant for tropical conditions.

The medicinal effects of guarana are believed to result from the high content of guaranine, associated alkaloids and tannin.

It has been suggested that the long-lasting effects of guarana occur because of the other components of the seeds.

Future research may show that various saponins also play an important part in the herb's pharmacology.

Guaranine and the other alkaloids have muscle-relaxant and diuretic properties. Guaranine is an alkaloid similar to the thein of tea and caffeine of coffee.

Case studies have indicated that guarana acts in a different way from caffeine and produces none of the undesirable side-effects.

Results of a trial comparing guarana and caffeine found that guarana had a strong and consistent positive effect on reported disposition and performance.

Guarana is useful for mild forms of diarrhea because it contains high tannin levels.

### Contradictions, Precautions, and Adverse Reactions

Contradictions: Hypersensitivity to any component. Not recommended for children, pregnant and breast-feeding women.

### Possible Side Effects

Guaranine, a chemical compound found in guarand, produces many of the same effects as caffeine. Individuals with conditions that may be complicated by high caffeine intake (including insomnia, depression, and pregnancy) should consult with a physician before adding guaraná to their diet.

### Dosage

Guaraná products that provide up to 400 milligrams of caffeine per day are considered safe.

Precautions: Same as Caffeine.

Adverse reactions: Same as Caffeine.

Interactions: It contains less than 4% of the alkaloid caffeine (as guaranine) and therefore the general drug interaction for caffeine should be considered.

Over-dosage: The legal amount not to exceed is 12 mcg/ml of caffeine in the urine.

Dosage and administration: Reports of use range anywhere from 500 to 1,000 mg, taken up to 3 times per day.

### Korean Ginseng

### Description

Korean ginseng is a deciduous perennial herb that reaches a height of 60 to 80 cm, with typical light colored fleshy root. The taste of the Korean ginseng root is sweetish at first but with a bitter aftertaste. The leaves are dark green colored and oval shaped. Each leave consists of five leaflets, the three terminal leaflets are larger than the two lateral ones. The ginseng stem is erect and deep red colored. Korean ginseng gives small red berries. The main active ingredients of Korean ginseng are ginsenosides. These steroid-like phytochemicals have adaptogenic properties, which give ginseng property to counter the effects of stress. The total ginsenoside content of a 6 year old root varies between 0.7 and 3%. The glycosides act on the adrenal glands, helping to prevent adrenal hypertrophy and excess corticosteroid production in response to stress. Ginsenosides increase protein synthesis and activity of neurotransmitters in the brain. Ginseng stimulates the formation of blood vessel and improves blood circulation in the brains, thereby improving memory and cognitive abilities. Ginseng is also used for diabetes, migraine, infections, radiation and chemotherapy protection, to aid in sleep, and to stimulate the appetite. Korean ginseng contains steroids such as panaxtriol. The steroids are remarkably similar in structure to anabolic steroids found naturally in our body. This makes Korean ginseng ideal for athletes and body builders looking for a natural alternative to anabolic steroid. Korean ginseng is also used by women for treatment of post menopausal symptoms.

Korean ginseng has had a long and illustrious history as an herb for health, and has been used for thousands of years throughout the Orient as a medicine and tonic. Early Chinese medicine texts written in the first century A.D. mention ginseng, and ginseng has long been classified by Chinese medicine as a “superior” herb. This means it is said to promote longevity and vitality. Legends around the world have touted ginseng as an aphrodisiac and sexual tonic.

Because of the number of herbs sold under the name of ginseng, there can be some confusion. Korean ginseng is a member of the Araliaceae family of plants, which also includes closely related American ginseng (Panax quinquefolius) and Siberian ginseng (Eleutherococcus senticosus). Both American and Siberian ginseng are considered by Chinese herbalists to be different herbs than Korean ginseng, and are said to have different effects and healing properties in the body. To add more confusion, there are eight herbs in Chinese medicine which are sometimes called ginseng, including black ginseng, purple ginseng, and prince's ginseng, some of which are not at all botanically related to Panax ginseng, so consumers should choose ginseng products with awareness.

### Actions and Pharmacology

### Actions:

The applicable part of Panax ginseng is the root. Panax ginseng contains several active constituents. The constituents thought to be of most importance are triterpenoid saponins referred to collectively as ginsenosides or panaxosides. Ginsenosides is the term developed by Asian researchers, and the term panaxosides was developed by early Russian researchers. Numerous subtypes of ginsenosides have been identified. Other constituents include pectin, B vitamins, and various flavonoids. Panax ginseng also contains the peptidoglycans, panaxans, which have hypoglycemic effects.

### Mechanism of Action:

The ginsenosides have a wide range of pharmacological activity and effects. In some cases, these isolated constituents seem to counteract each other's activity. For example, ginsenoside Rg1, raises blood pressure and acts as a central nervous system (CNS) stimulant. Ginsenoside Rb1 lowers blood pressure and acts as a CNS depressant. They also seem to interfere with platelet aggregation and coagulation. Ginsenosides also potentiate nerve growth factor and might confer neuroprotection through nicotinic activity. There is also evidence that ginsenosides can relax human bronchial smooth muscle by stimulating the release of nitrous oxide from airway epithelium which may account for the potential anti-asthmatic effect of Panax ginseng. However, research on related ginseng species, Panax pseudoginseng, suggests these ginsenosides may not be pharmacologically significant. Rb1 has a low oral bioavailability, and Rg1 is rapidly eliminated from the blood in animal models. Ginseng is widely used as a general tonic or “adaptogen” for fighting stress. There is some evidence that it might work against stress by affecting the hypothalamic-pituitary-adrenal (HPA) axis. Panax ginseng saponins seem to increase serum cortisol concentrations. Panax ginseng might also increase dehydroepiandrosterone sulfate (DHEA-S) levels in women. Panax ginseng might affect immune function and might have anticancer effects. Panax ginseng appears to stimulate natural-killer cell activity and possibly other immune-system activity. It might also have some antitumor activity. Extracts of Panax ginseng decrease the production of tumor necrosis factor (TNF), diminish DNA strand breakage, and inhibit the formation of induced skin tumors. There is conflicting research about the antioxidant and free radical scavenging activity of panax ginseng. Ginsenosides have been shown to inhibit tumor cell invasion and suppress sister chromatid exchanges in human lymphocytes. Panax ginseng also contains water insoluble polyacetylenic constituents such as panaxynol, panaxydol, and panaxytriol. Panaxydol seems to have antiproliferative effects on various types of cancer cells by inhibiting cancer cell growth at the cell cycle G1 to S transition phase. In peptic ulceration, Panax ginseng has shown inhibitory activity on Helicobacter pylori-induced hemagglutination. Samgyetang, a soup made from chicken, panax ginseng, garlic, jujube, and chestnuts, appears to offer protection from experimentally induced peptic ulcers. Panax ginseng may lower serum cholesterol and triglycerides, possibly by increasing lipoprotein lipase activity, which enhances lipid metabolism. However, panax ginseng appears to have negligible effects on cardiovascular function. Some ginsenosides have structural similarities to cardiac glycosides and can interfere with measurement of serum digoxin levels by some assay methods. It is not clear whether panax ginseng has any of the pharmacological effects of cardiac glycosides. Panax ginseng may affect blood glucose. Preliminary evidence that Panax ginseng might reduce tissue insulin resistance and changes in gene expression in Type II diabetes. Ginsenosides in Panax ginseng might also directly stimulate insulin release. The effect of various ginsengs on glucose appears to be related in part to the mix of ginsenosides. Other nonginsenoside constituents likely affect blood glucose as well. Panax ginseng and other ginsengs contain protopanaxadiol (PPD) ginsenosides, Rb1, Rb2, Rc, and Rd. They also contain protopanaxatriol (PPT) ginsenosides, Rg1, Re, and Rf. A higher ratio of PPD ginsenosides to PPT ginsenosides is related to greater blood glucose and insulin lowering potency of the ginseng product. Compared with American ginseng, panax ginseng appears to have a lower PPD to PPT ratio and may have less blood glucose.

Pharmacokinetics Not documented.

Indications and use Insufficient literary information available.

### Research Summary

Scientists have isolated what they believe are the primary active ingredients in ginseng, chemicals termed saponin triterpenoid glycosides, or commonly called ginsenocides. There are nearly 30 ginsenocides in Korean ginseng. Much research on Korean ginseng has been conducted in China, but controlled human experiments with it have not been easily accessible to the English-speaking world. Recent research in China was summarized by Dr. C. Lui in the February 1992 issue of the Journal of Ethnopharmacology, where he wrote that Panax ginseng was found to contain 28 ginsenocides that “act on the central nervous system, cardiovascular system and endocrine secretion, promote immune function, and have effects on anti-aging and relieving stress.”

A study performed over three years in Germany showed athletes given ginseng had favorable improvement in several categories over a control group who took a placebo. Another 1982 study showed that athletes given ginseng had improved oxygen intake and faster recovery time than those given placebos.

In general, studies show that ginseng enhances mental performance, learning time, and memory. One study of sixteen volunteers showed improvement on a wide variety of mental tests, including mathematics. Another study showed that those performing intricate and mentally demanding tasks improved performance when given Korean ginseng. Finally, a study has shown improvement of mood in depression sufferers with the use of ginseng.

Patients with chronic fatigue who were given ginseng showed a statistically significant improvement in physical tests and in mental attention and concentration, when compared with those given placebos.

Animal studies have shown that ginseng can facilitate the release of insulin from the pancreas and increase the number of insulin receptors in the body.

Scientific analysis of ginseng has shown that it has antioxidant effects, similar to the effects of vitamins A, C, and E. Thus, ginseng could be beneficial in combating the negative effects of pollution, radiation, and aging.

Some studies have shown that Korean ginseng reduces total cholesterol and increases levels of good cholesterol in the body.

Several tests have shown that Korean ginseng increases immune cell activity in the body, including the activity of T-cells and lymphocytes, which are instrumental in fighting cancer and other immune system disorders like AIDS. A Korean study indicates that taking ginseng may reduce the chances of getting cancer, as a survey of more than 1,800 patients in a hospital in Seoul showed that those who did not have cancer were more likely to have taken ginseng regularly than those patients who had contracted cancer.

One study showed significant improvement in an elderly test group in visual and auditory reaction time and cardiopulmonary function when given controlled amounts of Korean ginseng. Korean ginseng has also been shown to alleviate symptoms of menopause.

Studies of human sexual function and Korean ginseng have been generally inconclusive, despite the wide acclaim of ginseng as a sexual tonic. Tests with lab animals and ginseng have shown some interesting results, indicating that Korean ginseng promotes the growth of male reproductive organs, increases sperm and testoterone levels, and increases sexual activity in laboratory animals. In general, scientists believe the link between ginseng and sex drive is due to ginseng's effect of strengthening overall health and balancing the hormonal system.

Contradictions, Precautions, and adverse reactions

### Contradictions:

### Precautions:

There are different kinds of ginseng. Red Korean ginseng is considered stronger and more stimulating than white, wild ginseng is stronger than cultivated, and Korean ginseng is generally believed to be slightly stronger than Chinese. Furthermore, American and Siberian ginseng have slightly different properties than Korean ginseng, and consumers should make an informed choice as to which herb is best suited for them. Chinese herbalists do not recommend Korean ginseng for those people who have “heat” disorders in their bodies, such as ulcers, high blood pressure, tension headaches, and symptoms associated with high stress levels. Korean ginseng is generally not recommended for those with symptoms of nervousness, mental imbalance, inflammation, or fever. Korean ginseng is not recommended for pregnant or lactating women, and women of childbearing age should use ginseng sparingly, as some studies imply that it can influence estrogen levels. Also, Chinese herbalists typically only prescribe ginseng to older people or the weak, as they believe that younger and stronger people do not benefit as much from it and ginseng is “wasted on the young.”

Because of the number of and demand for ginseng products on the market, consumers should search for a reputable brand, preferably with a standardized percentage of active ingredients. To illustrate the mislabeling found with some ginseng products, Consumer Reports magazine analyzed ten nationally-distributed ginseng products in 1995. They found that several of them lacked significant amounts of ginsenocides, despite claims on the packaging to the contrary. Ginseng fraud has led the American Botanical Council, publisher of HerbalGram magazine, to initiate the Ginseng Evaluation Program, a comprehensive study and standardization of ginseng products on the American market. This study and its labeling standards are still under development, and consumers should watch for it.

### Adverse Reactions:

Korean ginseng acts as a slight stimulant in the body, and in some cases can cause overstimulation, irritability, nervousness and insomnia, although strong side effects are generally rare. Taking too high a dosage of ginseng, or taking ginseng for too long without a break, can cause ginseng intoxication, for which symptoms might include headaches, insomnia, seeing spots, dizziness, shortage of breath and gastrointestinal discomfort. Long term use may cause menstrual abnormalities and breast tenderness in some women.

### Interactions:

Those taking hormonal drugs should use ginseng with care. Ginseng should not be taken with caffeine or other stimulants as these may increase its stimulatory effects and cause uncomfortable side effects.

Over-dosage: Not Documented

Dosage and administration: 500 mg-1 g per day.

### Manufacturing

We design quality into our products. Areas of the manufacturing process that require control are identified during the development of the product, and the effects of variables and appropriate limits are established.

All manufacturing personnel have access to bathroom and cafeteria, which cannot be directly accessed from the manufacturing area. The employees have direct access to warehousing and final packaging area. Employees have to go through an airlock and dress properly before entering the manufacturing area.

The manufacturing area, its floor, walls, and sealing are constructed in such a way that no holes or cracks are possible.

Once R&D development is completed and ready to transfer to commercial manufacturing, the R&D project manager will submit to QSD for product transfer. The product transfer consists of QSD commercial production documentation (work order, specifications sheet, etc.) including employees' training and implementation.

The commercial manufacturing consists of many steps, controlled and released by the QSD.

### SEQUENCE LISTING

Not Applicable

### REFERENCES CITED

U.S. Pat. Documents

| | |
|---|---|
| 6,428,824 | 2002 |
| 6,267,995 | 2001 |
| 6,552,206 | 2003 |
| 5,302,611 | 1994 |
| 4,844,901 | 1989 |
| 4,940,725 | 1990 |

### CROSS REFERENCES TO RELATED APPLICATIONS

Valentova K, Ulrichova J. Smallanthus sonchifolius and Lepidium meyenii—prospective Andean crops for the prevention of chronic diseases. Biomed Pap Med Fac Univ Palacky Olomouc Czech Repub 2003; 147:119-30.
Smith E. Maca root: Modern rediscovery of an ancient Andean fertility food. J Amer Herbalists Guild 2003; 4:15-21.
Piacente S, Carbone V, Plaza A, Zampelli A, Pizza C. Investigation of the tuber constituents of maca (Lepidium meyenii Walp.). J Agric Food Chem 2002; 50:5621-5.

Gonzales G F, Cordova A, Vega K, et al. Effect of Lepidium meyenii (maca) on sexual desire and its absent relationship with serum testosterone levels in adult healthy men. Andrologia 2002; 34:367-72.
Gonzales G F, Cordova A, Vega K, et al. Effect of Lepidium meyenii (maca), a root with aphrodisiac and fertility-enhancing properties, on serum reproductive hormone levels in adult healthy men. J Endocrinol 2003; 176:163-8.
Gonzales G F, Cordova A, Gonzales C, et al. Lepidium meyenii (maca) improved semen parameters in adult men. Asian J Androl 2001; 3:301-3.
Cicero A F, Piacente S, Plaza A, et al. Hexanic maca extract improves rat sexual performance more effectively than methanolic and chloroformic maca extracts. Andrologia 2002; 34:177-9.
Gonzales G F, Ruiz A, Gonzales C, et al. Effect of Lepidium meyenii (maca) roots on spermatogenesis of male rats. Asian J Androl 2001; 3:231-3.
Ruiz-Luna A C, Salazar S, Aspajo N J, et al. Lepidium meyenii (maca) increases litter size in normal adult female mice. Reprod Biol Endocrinol 2005; 3:16.
Lopez-Fando A, Gomez-Serranillos M P, Iglesias I, et al. Lepidium peruvianum Chacon restores homeostasis impaired by restraint stress. Phytother Res 2004; 18:471-4.
Gonzales G F, Miranda S, Nieto J, et al. Red maca (Lepidium meyenii) reduced prostate size in rats. Reprod Biol Endocrinol 2005; 3:5.
Gonzales G F, Cordova A, Gonzales C, et al. Lepidium meyenii (maca) improved semen parameters in adult men. Asian J Androl 2001; 3:301-3.
Aguilar J L, Rojas P, Marcelo A, et al. Anti-inflammatory activity of two different extracts of Uncaria tomentosa (Rubiaceae). J Ethnopharmacol 2002; 81(2):271-276.
Akesson C, Lindgren H, Pero R W, et al. An extract of Uncaria tomentosa inhibiting cell division and NF-kappa B activity without inducing cell death. Int. Immunopharmacol. 2003; 3(13-14):1889-1900.
Allen-Hall L, Cano P, Amason J T, et al. Treatment of THP-1 cells with Uncaria tomentosa extracts differentially regulates the expression if IL-1beta and TNF-alpha. J Ethnopharmacol 1-19-2007; 109(2):312-317.
Ccahuana-Vasquez R A, Santos S S, Koga-Ito C Y, et al. Antimicrobial activity of Uncaria tomentosa against oral human pathogens. Braz. Oral Res 2007; 21(1):46-50.
Cheng A C, Jian C B, Huang Y T, et al. Induction of apoptosis by Uncaria tomentosa through reactive oxygen species production, cytochrome c release, and caspases activation in human leukemia cells. Food Chem Toxicol 2007; 45(11):2206-2218.
Garcia Prado E, Garcia Gimenez M D, De la Puerta V, et al. Antiproliferative effects of mitraphylline, a pentacyclic oxindole alkaloid of Uncaria tomentosa on human glioma and neuroblastoma cell lines. Phytomedicine 2007; 14(4):280-284.
Hardin S R. Cat's claw: an Amazonian vine decreases inflammation in osteoarthritis. Complement Ther Clin Pract. 2007 February; 13(1):25-8.
Kitajima M, Hashimoto K, Yokoya M, et al. Two new nor-triterpene glycosides from peruvian “Una de Gato” (Uncaria tomentosa). J. Nat. Prod. 2003; 66(2):320-323.
Lee J, Son D, Lee P, et al. Alkaloid fraction of Uncaria rhynchophylla protects against N-methyl-D-aspartate-induced apoptosis in rat hippocampal slices. Neurosci. Lett. 9-4-2003; 348(1):51-55.
Lee J, Son D, Lee P, et al. Protective effect of methanol extract of Uncaria rhynchophylla against excitotoxicity induced by N-methyl-D-aspartate in rat hippocampus. J Pharmacol Sci. 2003; 92(1):70-73.
Moreno S R, Silva A L, Dire G, et al. Effect of oral ingestion of an extract of the herb Uncaria tomentosa on the biodistribution of sodium pertechnetate in rats. Braz. J Med Biol Res 2007; 40(1):77-80.
Mur E, Hartig F, Eibl G, et al. Randomized double blind trial of an extract from the pentacyclic alkaloid-chemotype of uncaria tomentosa for the treatment of rheumatoid arthritis. J Rheumatol 2002; 29(4):678-681.
Piscoya J, Rodriguez Z, Bustamante S A, et al. Efficacy and safety of freeze-dried cat's claw in osteoarthritis of the knee: mechanisms of action of the species Uncaria guianensis. Inflamm. Res 2001; 50(9):442-448.
Setty A R, Sigal L H. Herbal medications commonly used in the practice of rheumatology: mechanisms of action, efficacy, and side effects. Semin. Arthritis Rheum 2005; 34(6):773-784.
Valerio L G Jr, Gonzales G F. Toxicological aspects of the South American herbs cat's claw (Uncaria tomentosa) and Maca (Lepidium meyenii): a critical synopsis. Toxicol. Rev 2005; 24(1):11-35.
Arpornsuwan T, Punjanon T. Tumor cell-selective antiproliferative effect of the extract from Morinda citrifolia fruits. Phytother Res 2006; 20(6):515-517.
Burrowes J D, Van Houten G. Use of alternative medicine by patients with stage 5 chronic kidney disease. Adv. Chronic. Kidney Dis. 2005; 12(3):312-325.
Carr M E, Klotz J, Bergeron M. Coumadin resistance and the vitamin supplement “Noni”. Am. J. Hematol. 2004; 77(1):103.
Hornick C A, Myers A, Sadowska-Krowicka H, et al. Inhibition of angiogenic initiation and disruption of newly established human vascular networks by juice from Morinda citrifolia (noni). Angiogenesis. 2003; 6(2): 143-149.
Jensen C J, Westendorf J, Wang M Y, et al Noni juice protects the liver. Eur J. Gastroenterol. Hepatol. 2006; 18(5):575-577.
Kamiya K, Tanaka Y, Endang H, et al. Chemical constituents of Morinda citrifolia fruits inhibit copper-induced low-density lipoprotein oxidation. J. Agric. Food Chem. 9-22-2004; 52(19):5843-5848.
Kamiya K, Tanaka Y, Endang H, et al. New anthraquinone and iridoid from the fruits of Morinda citrifolia. Chem Pharm Bull (Tokyo) 2005; 53(12):1597-1599.
Langford J, Doughty A, Wang M, et al. Effects of Morinda citrifolia on quality of life and auditory function in postmenopausal women. J. Altern. Complement Med 2004; 10(5):737-739.
Li R W, Myers S P, Leach D N, et al. A cross-cultural study: anti-inflammatory activity of Australian and Chinese plants. J Ethnopharmacol 2003; 85(1):25-32.
Millonig G, Stadlmann S, Vogel W. Herbal hepatotoxicity: acute hepatitis caused by a Noni preparation (Morinda citrifolia). Eur. J. Gastroenterol. Hepatol. 2005; 17(4):445-447.
Pawlus A D, Su B N, Keller W J, et al. An anthraquinone with potent quinone reductase-inducing activity and other constituents of the fruits of Morinda citrifolia (noni). J Nat Prod 2005; 68(12):1720-1722.
Pu H F, Huang W J, Tseng W M, et al. Effects of juice from Morinda citrifolia (Noni) on gastric emptying in male rats. Chin J Physiol 12-31-2004; 47(4):169-174.
Shotipruk A, Kiatsongserm J, Pavasant P, et al. Pressurized hot water extraction of anthraquinones from the roots of Morinda citrifolia. Biotechnol. Prog. 2004; 20(6):1872-1875.
Stadlbauer V, Fickert P, Lackner C, et al. Hepatotoxicity of NONI juice: Report of two cases. World J. Gastroenterol. 8-14-2005; 11(30):4758-4760.
Su B N, Pawlus A D, Jung H A, et al. Chemical constituents of the fruits of Morinda citrifolia (Noni) and their antioxidant activity. J Nat Prod 2005; 68(4):592-595.
Ito Y, Wakai K, Suzuki K, et al. Lung cancer mortality and serum levels of carotenoids, retinol, tocopherols, and folic acid in men and women: a case-control study nested in the JACC Study. J. Epidemiol. 2005; 15 Suppl 2:S140-S149.
Koh H H, Murray U, Nolan D, et al. Plasma and macular responses to lutein supplement in subjects with and without age-related maculopathy: a pilot study. Exp. Eye Res. 2004; 79(1):21-27.
Lietz G, Mulokozi G, Henry J C, et al. Xanthophyll and hydrocarbon carotenoid patterns differ in plasma and breast milk of women supplemented with red palm oil during pregnancy and lactation. J Nutr 2006; 136(7):1821-1827.
Olmedilla B, Granado F, Blanco I, et al. Lutein, but not alpha-tocopherol, supplementation improves visual function in patients with age-related cataracts: a 2-y double-blind, placebo-controlled pilot study. Nutrition 2003; 19(1):21-24.
Richer S, Stiles W, Statkute L, et al. Double-masked, placebo-controlled, randomized trial of lutein and antioxidant supplementation in the intervention of atrophic age-related macular degeneration: the Veterans LAST study (Lutein Antioxidant Supplementation Trial). Optometry. 2004; 75(4):216-230.
Sesso H D, Buring J E, Norkus E P, et al. Plasma lycopene, other carotenoids, and retinol and the risk of cardiovascular disease in men. Am J Clin Nutr 2005; 81(5):990-997.
Shao A, Hathcock J N. Risk assessment for the carotenoids lutein and lycopene. Regul. Toxicol Pharmacol 2006; 45(3):289-298.
Thurmann P A, Schalch W, Aebischer J C, et al. Plasma kinetics of lutein, zeaxanthin, and 3-dehydro-lutein after multiple oral doses of a lutein supplement. Am J Clin Nutr 2005; 82(1):88-97.
Tulley R T, Vaidyanathan J, Wilson J B, et al. Daily intake of multivitamins during long-term intake of olestra in men prevents declines in serum vitamins A and E but not carotenoids. J Nutr 2005; 135(6):1456-1461.
Whitehead A J, Mares J A, Danis R P. Macular pigment: a review of current knowledge. Arch Ophthalmol. 2006; 124(7):1038-1045.
Zhao X, Aldini G, Johnson E J, et al. Modification of lymphocyte DNA damage by carotenoid supplementation in postmenopausal women. Am J Clin Nutr 2006; 83(1):163-169.
Brown M J, Ferruzzi M G, Nguyen M L, et al. Carotenoid bioavailability is higher from salads ingested with full-fat than with fat-reduced salad dressings as measured with electrochemical detection. Am J Clin Nutr 2004; 80(2):396-403.
Clark P E, Hall M C, Borden L S Jr, et al. Phase I-II prospective dose-escalating trial of lycopene in patients with biochemical relapse of prostate cancer after definitive local therapy. Urology. 2006 June; 67(6):1257-61.
Gianetti J, Pedrinelli R, Petrucci R, et al. Inverse association between carotid intima-media thickness and the antioxidant lycopene in atherosclerosis. Am Heart J 2002; 143(3):467-474.
Giovannucci E, Clinton S K. Tomatoes, lycopene, and prostate cancer. Proc Soc Exp Biol Med 1998; 218(2):129-139.
Giovannucci E. Tomatoes, tomato-based products, lycopene, and cancer: review of the epidemiologic literature. J Natl Cancer Inst 1999; 91(4):317-331.
Giovannucci E, Rimm E B, Liu Y, et al. A prospective study of tomato products, lycopene, and prostate cancer risk. J Natl Cancer Inst 2002; 94(5):391-398.
Gupta N P, Kumar R. Lycopene therapy in idiopathic male infertility—a preliminary report. Int Urol Nephrol 2002; 34(3):369-372.
Hadley C W, Clinton S K, Schwartz S J. The consumption of processed tomato products enhances plasma lycopene concentrations in association with a reduced lipoprotein sensitivity to oxidative damage. J Nutr 2003; 133(3):727-732.
Sharma J B, Kumar A, Kumar A, et al. Effect of lycopene on pre-eclampsia and intra-uterine growth retardation in primigravidas. Int J Gynaecol Obstet 2003; 81(3):257-262.
Singh M, Krishanappa R, Bagewadi A, et al. Efficacy of oral lycopene in the treatment of oral leukoplakia. Oral Oncol 2004; 40(6):591-596.
Watzl B, Bub A, Blockhaus M, et al. Prolonged tomato juice consumption has no effect on cell-mediated immunity of well-nourished elderly men and women. J Nutr 2000; 130(7):1719-1723.
Endoh D, Okui T, Ozawa S, et al. Protective effect of a lignan-containing flaxseed extract against CCl(4)-induced hepatic injury. J. Vet. Med. Sci. 2002; 64(9):761-765.
Goss P E, Li T, Theriault M, et al. Effects of dietary flaxseed in women with cyclical mastalgia. Breast Cancer Res Treat 2000; 64:49.
Hu F B, Stampfer M J, Manson J E, et al. Dietary intake of alpha-linolenic acid and risk of fatal ischemic heart disease among women. Am J Clin Nutr 1999; 69(5):890-897.
Joshi K, Lad S, Kale M, et al. Supplementation with flax oil and vitamin C improves the outcome of Attention Deficit Hyperactivity Disorder (ADHD). Prostaglandins Leukot Essent Fatty Acids. 2006 January; 74(1):17-21.
Lemay A, Dodin S, Kadri N, et al. Flaxseed dietary supplement versus hormone replacement therapy in hypercholesterolemic menopausal women. Obstet. Gynecol. 2002; 100(3):495-504.
Lin X, Gingrich J R, Bao W, et al. Effect of flaxseed supplementation on prostatic carcinoma in transgenic mice. Urology 2002; 60(5):919-924.
Newton M, Combest W, Kosier J H, et al. Selected herbal dietary supplements used to manage climacteric (menopausal-type) symptoms. Urol. Nurs. 2002; 22(4):267-272.
Oomen C M, Ocke M C, Feskens E J, et al. Alpha-Linolenic acid intake is not beneficially associated with 10-y risk of coronary artery disease incidence: the Zutphen Elderly Study. Am J Clin Nutr 2001; 74(4):457-463.
Stoll A L, Severus W E, Freeman M P, et al. Omega 3 fatty acids in bipolar disorder: a preliminary double-blind, placebo-controlled trial. Arch Gen. Psychiatry 1999; 56(5):407-412.
von Schacky C, Angerer P, Kothny W, et al. The effect of dietary omega-3 fatty acids on coronary atherosclerosis. A randomized, double-blind, placebo-controlled trial. Ann Intern Med Apr. 6, 1999; 130(7):554-562.
Young G S, Conquer J A, Thomas R. Effect of randomized supplementation with high dose olive, flax or fish oil on serum phospholipid fatty acid levels in adults with attention deficit hyperactivity disorder. Reprod Nutr Dev. 2005 September-October; 45(5):549-58.
Effects of soy lecithin phosphatidic acid and phosphatidylserine complex (PAS) on the endocrine and psychological responses to mental stress. Stress. 2004 June; 7(2):119-26.
Safety of soy-derived phosphatidylserine in elderly people. Nutr Neurosci. 2002 October; 5(5):337-43.
The influence of soy-derived phosphatidylserine on cognition in age-associated memory impairment. Jorisse B L. Nutr Neurosci 2001; 4(2):121-34
Experimental Psychopharmacology Unit, Brain & Behaviour Institute, Department of Psychiatry and Neuropsychology, Maastricht, The Netherlands.
Fraumeni J F Jr, Scotto J, Dunham L J. Coffee-drinking and bladder cancer. Lancet 11-27-1971; 2(7735):1204.
Fukumasu H, Silva T C, Avanzo J L, et al. Chemopreventive effects of Paullinia cupana Mart var. sorbilis, the guarana, on mouse hepatocarcinogenesis. Cancer Lett. 5-7-2005;
Haller C A. Jacob P, Benowitz N L. Short-term metabolic and hemodynamic effects of ephedra and guarana combinations. Clin Pharmacol.Ther. 2005; 77(6):560-571.
Hess A M, Sullivan D L. Potential for toxicity with use of bitter orange extract and guarana for weight loss. Ann. Pharmacother. 2005; 39(3):574-575.
Jacobson M F, Goldman A S, Syme R H. Coffee and birth defects. Lancet 6-27-1981; 1(8235):1415-1416.
James J E. Is habitual caffeine use a preventable cardiovascular risk factor? Lancet 1-25-1997; 349(9047):279-281.
Joesoef M R, Beral V, Rolfs R T, et al. Are caffeinated beverages risk factors for delayed conception? Lancet Jan. 20, 1990; 335(8682):136-137.
Nyska A, Murphy E, Foley J F, et al. Acute hemorrhagic myocardial necrosis and sudden death of rats exposed to a combination of ephedrine and caffeine. Toxicol.Sci. 2005; 83(2):388-396.
Roberts A T, Jonge-Levitan L, Parker C C, et al. The effect of an herbal supplement containing black tea and caffeine on metabolic parameters in humans. Altern Med Rev 2005; 10(4):321-325.
Shapiro P. Caffeine for allergic rhinitis. Lancet Apr. 3, 1982; 1(8275):793.
Antistress effects of bacosides of Bacopa monnieri Phytother Res. 2002 November; 16(7):639-45.
Bacopasides III-V: three new triterpenoid glycosides from Bacopa monniera. Chem Pharm Bull (Tokyo). 2003 February; 51(2):215-7.
Broncho-vasodilatory activity of fractions and pure constituents isolated from Bacopa monniera. J. Ethnopharmacol. 2003 May; 86(1):27-35.
In vitro evaluation of Bacopa monniera on anti-Helicobacter pylori activity and accumulation of prostaglandins. Phytomedicine. 2003; 10(6-7):523-7.
Free radical scavenging capacity and protective effect of Bacopa monniera on DNA damage. Phytother Res. 2003 September; 17(8):870-5.
Phytotoxic and antimicrobial constituents of Bacopa monnieri and Holmskioldia sanguinea. Phytother Res. 2004 February; 18(2):114-7.
Quantitative determination of the major saponin mixture bacoside A in Bacopa monnieri by HPLC. Phytochem Anal. 2005 January-February; 16(1):24-9.
Antidepressant activity of standardized extract of Bacopa monniera in experimental models of depression in rats. Phytomedicine. 2002 April; 9(3):207-11.
Effect of Bacopa monniera and Azadirachta indica on gastric ulceration and healing in experimental NIDDM rats. Indian J Exp Biol. 2004 April; 42(4):389-97.
Adaptogenic effect of Bacopa monniera (Brahmi). Pharmacol Biochem Behay. 2003 July; 75(4):823-30
The acute effects of an extract of Bacopa monniera (Brahmi) on cognitive function in healthy normal subjects. Hum Psychopharmacol. 2001 June; 16(4):345-351.
The chronic effects of an extract of Bacopa monniera (Brahmi) on cognitive function in healthy human subjects. Psychopharmacology (Berl) 2001 August; 156(4):481-4
Chronic effects of Brahmi (Bacopa monniera) on human memory. Neuropsychopharmacology. 2002 August; 27(2):279-81.
Examining the nootropic effects of a special extract of Bacopa monniera on human cognitive functioning: 90 day double-blind placebo-controlled randomized trial. Phytother Res. 2008 Aug. 6. Stough C, Downey L A, Lloyd J, Silber B, Redman S, Hutchison C, Wesnes K, Nathan P J. Brain Sciences Institute, Swinburne University, P.O. Box 218 (H99), Hawthorn, Victoria 3122, Australia

### STATEMENT REGARDING FEDERALLY SPONSORED RESEARCH OR DEVELOPMENT

Not Applicable

### NAMES OF THE PARTIES TO A JOINT RESEARCH AGREEMENT

Not Applicable

### INCORPORATION-BY-REFERENCE OF THE MATERIAL SUBMITTED ON COMPACT DISC

### Enclosed

We claim that:

1. A homeostasis inducing composition comprised of the following ingredients:
Lepidium meyenii (Maca), Croton planstigma (Dragon's blood) tree sap, Uncaria tomentosa (Cat's Claw), Morinda citrifolia (Noni fruit) 4:1 PE, Lutein, Lycopene 5%, Flaxseed Oil (Omega-3-Fatty Acids), Vinpocetine, Phosphatidyl Serine 50%, Korean Ginseng 80%, Bacopa monnieri (Bacopin), CDP Choline (Cognizin®), Guaranine (Guarana Seed PE 12%), Yerba Mate Ext. 8%—which is designed to promote overall well being fee from diseases.

2. A liquid composition of claim 1 comprised of the following ingredients:
Lepidium meyenii (Maca), Croton planstigma (Dragon's blood) tree sap, Uncaria tomentosa (Cat's Claw), Morinda citrifolia (Noni fruit) 4:1 PE, Lutein, Lycopene 5%, Flaxseed Oil (Omega-3-Fatty Acids), Vinpocetine, Phosphatidyl Serine 50%, Korean Ginseng 80%, Bacopa monnieri (Bacopin), CDP Choline (Cognizin®), Guaranine (Guarana Seed PE 12%), Yerba Mate Ext. 8%.

3. Give energy and vigor
A method for giving energy and vigor, comprising administering an effective amount of the composition of claim 1 in form of liquid of claim 2 to a person in need of lifeforce.
a. Lifeforce liquid help to induce optimal health and state of balance by:
b. Administered by liquid form with an effective amount of composition of claim 1.
c. Administered by liquid form with an effective amount of composition of claim 2.

4. Induce homeostasis
A method for returning the body to homeostasis, comprising administering an effective amount of the composition of claim 1 in form of liquid of claim 2 to a person in need of homeostasis
a. Lifeforce liquid help to induce optimal health and state of balance by:
b. Administered by liquid form with an effective amount of composition of claim 1.
c. Administered by liquid form with an effective amount of composition of claim 2.
